SEQUENCE LISTING (51 sequences)

SEQ ID NO: 1 - protein, 68 aa, Bacteriophage P21
MDKISTGIAYGTSAGSAGYWFLQWLDQVSPSQWAAIGVLGSLVLGFLTYLTNLYFKIREDRRKAARGE

SEQ ID NO: 2 - protein, 71 aa, Bacteriophage P21
MKSMDKISTGIAYGTSAGSAGYWFLQWLDQVSPSQWAAIGVLGSLVLGFLTYLTNLYFKIREDRRKAARGE

SEQ ID NO: 3 - protein, 71 aa, Artificial Sequence
  Mutant of S21-71 anti-pinholin of bacteriophage P21
MKSADKISTGIAYGTSAGSAGYWFLQWLDQVSPSQWAAIGVLGSLVLGFLTYLTNLYFKIREDRRKAARGE

SEQ ID NO: 4 - protein, 41 aa, Artificial Sequence
  Mutant of S21-68 pinholin of bacteriophage P21
MSGSMWAAIGVLGSLVLGFLTYLTNLYFKIREDRRKAARGE

SEQ ID NO: 5 - protein, 53 aa, Artificial Sequence
  Mutant of S21-68 pinholin of bacteriophage P21
MSSSGGSETVRFQSGSMWAAIGVLGSLVLGFLTYLTNLYFKIREDRRKAARGE

SEQ ID NO: 6 - protein, 105 aa, Bacteriophage lambda
MPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQ

SEQ ID NO: 7 - protein, 107 aa, Bacteriophage lambda
MKMPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQ

SEQ ID NO: 8 - protein, 107 aa, Artificial Sequence
  Mutant of S107 anti-pinholin of bacteriophage lambda
MKAPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQ

SEQ ID NO: 9 - protein, 218 aa, Bacteriophage T4
MAAPRISFSPSDILFGVLDRLFKDNATGKVLASRVAVVILLFIMAIVWYRGDSFFEYYKQSKYETYSEIIEKERTARFESVALEQLQIVHISSEADFSAVYSFRPKNLNYFVDIIAYEGKLPSTISEKSLGGYPVDKTMDEYTVHLNGRHYYSNLKFAFLPTKKPTPEINYMYSCPYFNLDNIYAGTITMYWYRNDHISNDRLESICAQAARILGRAK

SEQ ID NO: 10 - protein, 48 aa, Artificial Sequence
  Mutant of T4 pinholin of bacteriophage T4
AAPRISFSPSDILFGVLDRLFKDNATGKVLASRVAVVILLFIMAIVWY

SEQ ID NO: 11 - protein, 16 aa, Artificial Sequence
  Mutant of T4 pinholin of bacteriophage T4
MVAVVILLFIMAIVWY

SEQ ID NO: 12 - protein, 82 aa, Chlorellavirus Next-to-Smith
MLLLIIHLSILVIFTAIYKMLPGGMFSNTDPTWVDCLYFSASTHTTVGYGDLTPKSPVAKLTATAHMLIVFAIVISSFTFPW

SEQ ID NO: 13 - protein, 82 aa, Artificial Sequence
  Mutant of KCVNTS channel
MLLLIIHLSILVIFTAIYKMLPGGMFSNTDPTWVDCLYFSASTHTTVGYGDLTPKSPVAKLTATAHMLIVFAIVISGFTFPW

SEQ ID NO: 14 - protein, 94 aa, Paramecium bursaria Chlorella virus 1
MLVFSKFLTRTEPFMIHLFILAMFVMIYKFFPGGFENNFSVANPDKKASWIDCIYFGVTTHSTVGFGDILPKTTGAKLCTIAHIVTVFFIVLTL

SEQ ID NO: 15 - protein, 49 aa, Escherichia coli
MKHNPLVVCLLIICITILTFTLLTRQTLYELRFRDGDKEVAALMACTSR

SEQ ID NO: 16 - protein, 29 aa, Escherichia coli
MNLVDIAILILKLIVAALQLLDAVLKYLK

SEQ ID NO: 17 - protein, 319 aa, Staphylococcus aureus
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 18 - protein, 36 aa, Artificial Sequence
  Mutant of Wza of E. coli
MAPLVRWNRVISQLVPTITGVHDLTETVRYIKTWPN

SEQ ID NO: 19 - protein, 33 aa, Influenza B virus
MLEPFQILSISSFILSALHFIAWTIGHLNQIKR

SEQ ID NO: 20 - protein, 37 aa, Artificial Sequence
  Transmembrane domain of TME1 of Hepatitis C virus
MIAGAHWGVLAGIAYFSMVGNWAKVLVVLLLFAGVDA

SEQ ID NO: 21 - protein, 31 aa, Artificial Sequence
  Transmembrane domain of TME2 of Hepatitis C virus
MEYVVLLFLLLADARVCSCLWMMLLISQAEA

SEQ ID NO: 22 - DNA, 38 bp, Artificial Sequence
  Primer
  misc_feature (15)..(16): n is a, c, g, or t
  misc_feature (18)..(19): n is a, c, g, or t
  misc_feature (21)..(22): n is a, c, g, or t
  misc_feature (25)..(26): n is a or c or g or t
  misc_feature (28)..(29): n is a or c or g or t
  misc_feature (31)..(32): n is a or c or g or t
tgatgaggcatatgnnknnknnkgacaaaatctcaact

SEQ ID NO: 23 - protein, 417 aa, Artificial Sequence
  R-GECO-1 Sensorprotein
MVDSSRRKWNKAGHAVRAIGRLSSPVVSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGPLPFAWDILSPQFMYGSKAYIKHPADIPDYFKLSFPEGFRWERVMNFEDGGIIHVNQDSSLQDGVFIYKVKLRGTNFPPDGPVMQKKTMGWEATRDQLTEEQIAEFKEAFSLFDKDGDGTITTKELGTVMRSLGQNPTEAELQDMINEVDADGDGTFDFPEFLTMMARKMNDTDSEEEIREAFRVFDKDGNGYIGAAELRHVMTDLGEKLTDEEVDEMIRVADIDGDGQVNYEEFVQMMTAK

SEQ ID NO: 24 - protein, 416 aa, Artificial Sequence
  G-GECO-1 Sensorprotein
MVDSSRRKWNKTGHAVRAIGRLSSLENVYIKADKQKNGIKANFKIRHNIEDGGVQLAYHYQQNTPIGDGPVLLPDNHYLSVQSILSKDPNEKRDHMVLLEFVTAAGITLGMDELYKGGTGGSMVSKGEELFTGVVPIQVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTRDQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYIGAAELRHVMTNLGEKLTDEEVDEMIRVADIDGDGQVNYEEFVQMMTAK

SEQ ID NO: 25 - protein, 523 aa, Artificial Sequence
  iGluSnFR Sensorprotein
MGSAAGSTLDKIAKNGVIVVGHRESSVPFSYYDNQQKVVGYSQDYSNAIVEAVKKKLNKPDLQVKLIPITSQNRIPLLQNGTFDFECGSTTNNVERQKQAAFSDTIFVVGTRLLTKKGGDIKDFANLKDKAVVVTSGTTSEVLLNKLNEEQKMNMRIISAKDHGDSFRTLESGRAVAFMMDDVLLAGERAKAKKPDNWEIVGKPQSQEAYGCMLRKDDPQFKKLMDDTIAQVQTSGEAEKWFDKWFKNPILVSHNVYIMADKQRNGIKANFKIRHNIEDGGVQLAYHYQQNTPIGDGPVLLPDNHYLSTQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNFNNPLNMNFELSDEMKALFKEPNDKALK

SEQ ID NO: 26 - DNA, 81 bp, Artificial Sequence
  T7.wt.sRBS Promoter Region
  promoter (1)..(19)
  misc_feature (20)..(44): Lac-Operator
  RBS (59)..(81)
taatacgactcactataggggaattgtgagcggataacaattcccctctagaaataattttgtttaactttaagaaggaga

SEQ ID NO: 27 - DNA, 63 bp, Artificial Sequence
  T7.100.sRBS Promoter Region
  promoter (1)..(19)
  misc_feature (20)..(44): Lac-Operator
  RBS (51)..(63)
taatacgactcactataggggaattgtgagcggataacaattccaccggtaaagaggagaaag

SEQ ID NO: 28 - protein, 67 aa, Artificial Sequence
  Truncation of S21-68 pinholin of bacteriophage P21
MKISTGIAYGTSAGSAGYWFLQWLDQVSPSQWAAIGVLGSLVLGFLTYLTNLYFKIREDRRKAARGE

SEQ ID NO: 29 - protein, 65 aa, Artificial Sequence
  Truncation of S21-68 pinholin of bacteriophage P21
MSTGIAYGTSAGSAGYWFLQWLDQVSPSQWAAIGVLGSLVLGFLTYLTNLYFKIREDRRKAARGE

SEQ ID NO: 30 - protein, 63 aa, Artificial Sequence
  Truncation of S21-68 pinholin of bacteriophage P21
MGIAYGTSAGSAGYWFLQWLDQVSPSQWAAIGVLGSLVLGFLTYLTNLYFKIREDRRKAARGE

SEQ ID NO: 31 - protein, 61 aa, Artificial Sequence
  Truncation of S21-68 pinholin of bacteriophage P21
MAYGTSAGSAGYWFLQWLDQVSPSQWAAIGVLGSLVLGFLTYLTNLYFKIREDRRKAARGE

SEQ ID NO: 32 - protein, 59 aa, Artificial Sequence
  Truncation of S21-68 pinholin of bacteriophage P21
MGTSAGSAGYWFLQWLDQVSPSQWAAIGVLGSLVLGFLTYLTNLYFKIREDRRKAARGE

SEQ ID NO: 33 - protein, 57 aa, Artificial Sequence
  Truncation of S21-68 pinholin of bacteriophage P21
MSAGSAGYWFLQWLDQVSPSQWAAIGVLGSLVLGFLTYLTNLYFKIREDRRKAARGE

SEQ ID NO: 34 - protein, 55 aa, Artificial Sequence
  Truncation of S21-68 pinholin of bacteriophage P21
MGSAGYWFLQWLDQVSPSQWAAIGVLGSLVLGFLTYLTNLYFKIREDRRKAARGE

SEQ ID NO: 35 - protein, 53 aa, Artificial Sequence
  Truncation of S21-68 pinholin of bacteriophage P21
MAGYWFLQWLDQVSPSQWAAIGVLGSLVLGFLTYLTNLYFKIREDRRKAARGE

SEQ ID NO: 36 - protein, 51 aa, Artificial Sequence
  Truncation of S21-68 pinholin of bacteriophage P21
MYWFLQWLDQVSPSQWAAIGVLGSLVLGFLTYLTNLYFKIREDRRKAARGE

SEQ ID NO: 37 - protein, 49 aa, Artificial Sequence
  Truncation of S21-68 pinholin of bacteriophage P21
MFLQWLDQVSPSQWAAIGVLGSLVLGFLTYLTNLYFKIREDRRKAARGE

SEQ ID NO: 38 - protein, 47 aa, Artificial Sequence
  Truncation of S21-68 pinholin of bacteriophage P21
MQWLDQVSPSQWAAIGVLGSLVLGFLTYLTNLYFKIREDRRKAARGE

SEQ ID NO: 39 - protein, 45 aa, Artificial Sequence
  Truncation of S21-68 pinholin of bacteriophage P21
MLDQVSPSQWAAIGVLGSLVLGFLTYLTNLYFKIREDRRKAARGE

SEQ ID NO: 40 - protein, 43 aa, Artificial Sequence
  Truncation of S21-68 pinholin of bacteriophage P21
MQVSPSQWAAIGVLGSLVLGFLTYLTNLYFKIREDRRKAARGE

SEQ ID NO: 41 - protein, 41 aa, Artificial Sequence
  Truncation of S21-68 pinholin of bacteriophage P21
MSPSQWAAIGVLGSLVLGFLTYLTNLYFKIREDRRKAARGE

SEQ ID NO: 42 - protein, 39 aa, Artificial Sequence
  Truncation of S21-68 pinholin of bacteriophage P21
MSQWAAIGVLGSLVLGFLTYLTNLYFKIREDRRKAARGE

SEQ ID NO: 43 - protein, 71 aa, Artificial Sequence
  Mutant of S21-71 anti-pinholin of bacteriophage P21
  MISC_FEATURE (2)..(2): Xaa can be Ala, Arg, Asn, Asp, Gln, Glu, Gly, His, Ile, Leu, Lys, Phe, Ser, Thr, Trp, Tyr or Val
  MISC_FEATURE (3)..(3): Xaa can be Ala, Arg, Asn, Asp, Gln, Glu, Gly, His, Ile, Leu, Lys, Phe, Ser, Thr, Trp, Tyr or Val
  MISC_FEATURE (4)..(4): Xaa can be Ala, Arg, Asn, Asp, Gln, Glu, Gly, His, Ile, Leu, Lys, Phe, Ser, Thr, Trp, Tyr or Val
MXXXDKISTGIAYGTSAGSAGYWFLQWLDQVSPSQWAAIGVLGSLVLGFLTYLTNLYFKIREDRRKAARGE

SEQ ID NO: 44 - protein, 71 aa, Artificial Sequence
  Mutant of S21-71 anti-pinholin of bacteriophage P21
MYLEDKISTGIAYGTSAGSAGYWFLQWLDQVSPSQWAAIGVLGSLVLGFLTYLTNLYFKIREDRRKAARGE

SEQ ID NO: 45 - protein, 71 aa, Artificial Sequence
  Mutant of S21-71 anti-pinholin of bacteriophage P21
MNFEDKISTGIAYGTSAGSAGYWFLQWLDQVSPSQWAAIGVLGSLVLGFLTYLTNLYFKIREDRRKAARGE

SEQ ID NO: 46 - protein, 71 aa, Artificial Sequence
  Mutant of S21-71 anti-pinholin of bacteriophage P21
MDWDDKISTGIAYGTSAGSAGYWFLQWLDQVSPSQWAAIGVLGSLVLGFLTYLTNLYFKIREDRRKAARGE

SEQ ID NO: 47 - protein, 71 aa, Artificial Sequence
  Mutant of S21-71 anti-pinholin of bacteriophage P21
MAGWDKISTGIAYGTSAGSAGYWFLQWLDQVSPSQWAAIGVLGSLVLGFLTYLTNLYFKIREDRRKAARGE

SEQ ID NO: 48 - protein, 71 aa, Artificial Sequence
  Mutant of S21-71 anti-pinholin of bacteriophage P21
MWRGDKISTGIAYGTSAGSAGYWFLQWLDQVSPSQWAAIGVLGSLVLGFLTYLTNLYFKIREDRRKAARGE

SEQ ID NO: 49 - protein, 71 aa, Artificial Sequence
  Mutant of S21-71 anti-pinholin of bacteriophage P21
MFQWDKISTGIAYGTSAGSAGYWFLQWLDQVSPSQWAAIGVLGSLVLGFLTYLTNLYFKIREDRRKAARGE

SEQ ID NO: 50 - DNA, 1096 bp, Artificial Sequence
  Transcription cassette in context of pCTRL2 with T7.wt.sRBS promoter
  terminator (1)..(72)
  terminator (85)..(132)
  promoter (158)..(176)
  misc_feature (177)..(201): Lac-Operator
  RBS (216)..(238)
  misc_feature (246)..(905): Coding sequence of mkO-Kappa fluorescent protein
  terminator (915)..(969)
  terminator (976)..(1022)
  terminator (1049)..(1096)
caaataaaacgaaaggctcagtcgaaagactgggcctttcgttttatctgttgtttgtcggtgaacgctctcgtcgacctgcagctagcataaccccttggggcctctaaacgggtcttgaggggttttttgccatgctgatgagccatgggctgcctaatacgactcactataggggaattgtgagcggataacaattcccctctagaaataattttgtttaactttaagaaggagatatacatatggtgtctgtaattaagccagaaatgaagatgcgttattacatggacggaagcgttaatggccatgagtttactatcgagggggagggaaccgggcgtccgtatgagggccaccaagaaatgacattacgcgttactatggctgagggtggacctatgccgtttgccttcgatctggtttcccacgtcttttgttacggccaccgtgtgttcacaaaatatcccgaagagatcccggactatttcaaacaggcgttcccagagggtcttagctgggaacgtagcctggaatttgaggacggaggatcagccagtgtatcggctcatatctctctgcgcggtaataccttctatcataagagcaaatttactggtgtaaactttcctgctgatggtccgattatgcaaaatcaaagtgtggactgggaaccatcgacagagaaaatcacagcctccgacggagtcctgaaaggagacgtaacgatgtacttaaagttagaagggggcggcaatcacaagtgccagtttaagacaacttacaaagcggctaaagaaattttggaaatgccgggtgaccactacatcgggcaccgccttgtacgtaagaccgagggtaatattactgaacaggtggaggatgcggtcgcccattcctaaggtaccggatccctagcataaccccgcggggcctcttcgggggtctcgcggggttttttgctgaaaggagctccaaataaaacgaaaggctcagtcgaaagactgggcctttcgttttatctcgagctgccaccgctgagcaataactagcataaccccttggggcctctaaacgggtcttgaggggttttttg

SEQ ID NO: 51 - DNA, 1071 bp, Artificial Sequence
  Transcription cassette in context of pCTRL2.T7.100.sRBS with T7.100.sRBS Promoter
  terminator (1)..(72)
  terminator (85)..(132)
  promoter (152)..(170)
  misc_feature (171)..(195): Lac-Operator
  RBS (202)..(214)
  misc_feature (221)..(880): Coding sequence of mkO-Kappa fluorescent protein
  terminator (890)..(944)
  terminator (951)..(997)
  terminator (1024)..(1071)
caaataaaacgaaaggctcagtcgaaagactgggcctttcgttttatctgttgtttgtcggtgaacgctctcgtcgacctgcagctagcataaccccttggggcctctaaacgggtcttgaggggttttttgccatgctgatgagccatggtaatacgactcactataggggaattgtgagcggataacaattccaccggtaaagaggagaaaggagcatatggtgtctgtaattaagccagaaatgaagatgcgttattacatggacggaagcgttaatggccatgagtttactatcgagggggagggaaccgggcgtccgtatgagggccaccaagaaatgacattacgcgttactatggctgagggtggacctatgccgtttgccttcgatctggtttcccacgtcttttgttacggccaccgtgtgttcacaaaatatcccgaagagatcccggactatttcaaacaggcgttcccagagggtcttagctgggaacgtagcctggaatttgaggacggaggatcagccagtgtatcggctcatatctctctgcgcggtaataccttctatcataagagcaaatttactggtgtaaactttcctgctgatggtccgattatgcaaaatcaaagtgtggactgggaaccatcgacagagaaaatcacagcctccgacggagtcctgaaaggagacgtaacgatgtacttaaagttagaagggggcggcaatcacaagtgccagtttaagacaacttacaaagcggctaaagaaattttggaaatgccgggtgaccactacatcgggcaccgccttgtacgtaagaccgagggtaatattactgaacaggtggaggatgcggtcgcccattcctaaggtaccggatccctagcataaccccgcggggcctcttcgggggtctcgcggggttttttgctgaaaggagctccaaataaaacgaaaggctcagtcgaaagactgggcctttcgttttatctcgagctgccaccgctgagcaataactagcataaccccttggggcctctaaacgggtcttgaggggttttttg

(19) United States
(12) Patent Application Publication
(10) Pub. No.: US 2023/0050056 A1
(43) Pub. Date: Feb. 16, 2023

(54) METHOD FOR SCREENING PORE-FORMING MEMBRANE PROTEINS, MEMBRANE TRANSPORTERS AND MOLECULAR SWITCHES

(71) Applicant: TECHNISCHE UNIVERSITÄT DARMSTADT, Darmstadt (DE)

(72) Inventors: Viktor STEIN, Darmstadt (DE); Wadim WEBER, Buettelborn (DE); Markus ROEDER, Darmstadt (DE)

(21) Appl. No.: 17/641,835

(22) PCT Filed: Sep. 11, 2020
(86) PCT No.: PCT/EP2020/075535

§ 371 Date: Sep. 19, 2022

(30) Foreign Application Priority Data
Sep. 11, 2019 (DE) 10 2019 124 459.5

(51) Int. Cl. G01N 33/68; C40B 30/00

(52) U.S. Cl. CPC G01N 33/6872; C40B 30/00

(57) ABSTRACT

The present invention relates to a method for screening pore-forming membrane proteins, membrane transporters and molecular switches. The invention also relates to a kit for carrying out the method.

The present invention relates to a method for screening pore-forming membrane proteins, membrane transporters and molecular switches. The invention also relates to a kit for carrying out the method.

### INTRODUCTION

The construction of proteins with customized structural and/or functional properties still relies on empirical testing of many different protein mutants due to the comparatively misunderstood relationship between sequence, structure and function of a protein. In practice, the testing of many individual protein mutants is achieved in comparatively short periods of time by means of so-called screening methods. In this case, a library of protein mutants is first tested for a desired property, selected and enriched. Subsequently, the mutants enriched in a desired function are sequenced and identified. In this case, it is critical to ensure physical association between genotype (i.e., the coding DNA, which can in each case be “read,” “described,” and “propagated”) and the phenotype (i.e., the function of a protein). In addition, in the development of novel screening methods, handling, throughput, sensitivity, dynamic range and quantitative and temporal resolution inter alia play a crucial role in investigating libraries of protein mutants as efficiently and flexibly as possible for a desired structural and/or functional property in practice.

The development of suitable screening methods in this case is found to be particularly demanding for (protein) nanopores: Especially since the expression of many membrane proteins, in particular of (protein) nanopores, is toxic and only a comparatively small number of screening methods have 30 been developed so far, which moreover suffer from a number of limitations.

Structural and functional properties of (protein) nanopores are traditionally investigated by electrophysiological techniques, predominantly by the technically demanding in-vitro bilayer technique. Here, the forming and opening of a (protein) nanopore, which is respectively integrated in a phospholipid bilayer, is measured by means of a current flow between two electrodes. Electrophysiological measuring methods are, however, limited to the testing of defined mutants in purified form and do not allow screening at high throughput. In this respect, a number of idiosyncratic systems have been developed, which however have a plurality of disadvantages:

(1) Growth-dependent screening methods, in particular in Escherichia coli, but also in other microorganisms, such as Saccharomyces cerevisiae, are based on the intrinsic toxicity of (protein) nanopores: Specifically, pore-forming membrane peptides and proteins depolarize the membrane potential and thus prevent growth. In the past, growth assays were successfully used, for example, to characterize pore-forming properties of individual membrane peptides (ACS Chemical Biology, 2016, 11, 910-20) or also to identify peptides, which could also inter alia have pore-forming properties, in combination by means of a screening method with an antibiotic effect (Cell, 2018, 172, 618-628).

Specific disadvantages: (i) poor temporal resolution of the pore-forming properties (e.g., hours to days in spot dilution assays (growth-based assays in E. coli require at least 12-16 hours, in contrast to the methods of the present invention, which as a function of the strength of expression and the pore-forming properties of a membrane peptide or membrane protein, generate a signal by 1-2 orders of magnitude more quickly ((in particular in the minute range)); (ii) minor differences in pore-forming properties can only be resolved to a limited extent; (iii) pore-forming properties cannot be selected positively, but only negatively. Growth-based screening methods thus always rely on technically demanding colony blotting or global analyses by means of high-cost next-generation DNA sequencing; (iv) toxicity due to pore-forming properties cannot be directly differentiated from other toxicity.

(2) In addition, a hemolytic assay for colonies and microtiter plates was developed: Specifically, pore-forming toxins, such as cyolysin A and fragatoxin C, are expressed in Escherichia coli, and the properties of individual mutants are subsequently visualized with addition of red blood cells, which result in digestion of the erythrocytes and the associated release of hemoglobin (JACS, 2013, 135, 13456-63; Angewandte Chemie [Applied Chemistry], 2016, 55, 12494-8).

Specific disadvantages: (i) Single-cell analysis is not possible by means of a flow cytometer, but requires co-incubation with erythrocytes either in the microtiter plate or on agar plates; (ii) The assay is limited to erythrocyte-specific toxins whose pore-forming properties are activated by sphingomyelin-containing lipid membranes.

(3) A genetic complementation assay based on Saccharomyces cerevisiae strains with deficient potassium channels. By recombination of optogenetic receptors with viral potassium channels, light-controlled potassium channels can be constructed (Science, 2015, 348, 707-10; Methods in Molecular Biology, 2017, 1596, 271-285).

Specific disadvantages: (i) similar to growth assays, poor temporal resolution of the pore-forming properties (e.g., hours to days in spot dilution assays); (ii) similar to growth assays, slight differences in properties can be resolved only to a limited extent both in terms of time and quantity.

(4) Liposome display refers to a screening method based on artificial cells. In a first step, cells surrounded by artificial, phospholipid bilayers are produced by means of a water-in-oil emulsion and centrifugation through a phospholipid bilayer. After an in vitro expression, in-vitro proteins incorporate autonomously into the membrane. The forming or opening of nanopores is examined while adding a dye, which diffuses into the interior of the artificial cell as a function of the (protein) nanopore and remains there by covalent conjugation with a tag (PNAS, 2013, 110, 16796-801; Nature Protocols, 2014, 9, 1578-91).

Specific disadvantages: (i) complex and technically demanding in-vitro procedure; (ii) folding and incorporation cannot be controlled by bacterial folding machines; (iii) low enrichment per selection round: e.g., 20 selection rounds were required to improve the functional insertion of alpha-hemolysin by a factor of 20.

(5) In principle, screening systems could be visualized both with voltage-dependent membrane dyes, such as DiSC3(5) or membrane-impermeable DNA-binding dyes, such as propidium iodide.

Specific disadvantages: (i) Synthetic dyes must be added externally and thus increase the complexity and cost of the assay; (ii) Propidium iodide has so far exclusively been used to investigate the effect of externally added peptides and has not yet been used to screen intracellularly expressed (protein) nanopores; that is to say, to what extent propidium iodide is suitable as a specific marker for the inner membrane, or propidium iodide is comparatively undifferentiated as a general marker of membrane integrity, is unclear; (iii) Propidium iodide is too large to get through defined pores of pinholins.

The construction of (protein) nanopores, channels and transporters with customized functions and properties has great potential for a number of biotechnological applications:

(a) In biosensor technology, as highly sensitive sensors that can detect clinically or biotechnologically relevant molecules in complex molecular environments.
(b) In non-invasive imaging, as genetically encoded reporters in order to specifically enrich low-molecular imaging substances (e.g., MRI-active reporter molecules) in individual cells and to thus mark them.
(c) In the construction of artificial biosynthesis and metabolic pathways as well as in chemical reaction technology, as membrane transporters that enable the import of substrates or the export of products through impermeable phospholipid membranes, for example in natural cells as well as biomimetic cell compartments.
(d) In preparation technology, in order to be able to clean specific molecules from complex environments (e.g., directly from the environment or chemical reaction compartments).

It is therefore an object of the invention to overcome the disadvantages of the prior art and to provide a correspondingly improved screening method and a kit for carrying out the method. The object is achieved by the subject matters of the claims.

Specifically, a screening method has been developed in order to measure and select structural and functional properties of a (protein) nanopore (e.g., forming or opening), preferably with a promiscuous permeability, in combination in various screening formats (e.g., in microtiter plates, by colony screening, and by means of flow cytometry), preferably at medium to high throughput of 10 10²-10⁸. At the molecular level, forming or opening a (protein) nanopore results in the inflow of a reporter substance into a cell; in turn, the inflow of the reporter substance is converted into a biophysical signal (e.g., fluorescence, bioluminescence or absorption) by means of a genetically encoded sensor protein and thus visualized. This in turn makes it possible to select the DNA sequence that encodes the desired pore-forming property and to read it by means of various DNA sequencing methods (e.g., according to the Sanger method or by means of DNA sequencing methods of the next generation), and moreover to further propagate and mutate it. In addition, the method can be used to screen either membrane transporters with specific permeabilities or molecular sensors/switches. The former includes the detection of a specific sensor that detects the inflow or outflow of a specific metabolic product. By the formation of non-specific pores (in particular in the inner membrane of E. coli), the latter enables the inflow or outflow of a specific metabolic product and thus positive-negative selection strategies at the level of individual cells at very high throughput by means of flow cytometry.

### DETAILED DESCRIPTION

The objects described above are achieved by the subject matters of the claims. In particular, the objects are achieved by a screening method comprising the following steps:

a) providing a cell and a medium surrounding the cell,

wherein the cell has a cell membrane impermeable to a reporter substance,

wherein the quotient of the concentration of the reporter substance in the interior of the cell and the concentration of the reporter substance in the medium surrounding the cell is at least 2 or at most 0.5;

b) detecting a signal that is dependent on the concentration of the reporter substance in the cell and that is generated with the aid of a sensor protein expressed in the cell;
c) inducing the expression of a membrane protein that increases the permeability of the cell membrane to the reporter substance;
d) detecting the signal that is dependent on the concentration of the reporter substance in the cell and that is generated with the aid of the sensor protein expressed in the cell;
e) calculating one or more comparative parameters from the signal strengths detected in steps b) and d), wherein step b) is carried out before step c) and wherein step d) is carried out after step c),
wherein steps a) to e) are carried out for at least two membrane proteins having different amino acid sequences and/or for at least two sensor proteins having different amino acid sequences.

The screening method of the present invention can be carried out in various formats. The method is preferably carried out in microtiter plates, by colony screening and/or by means of flow cytometry.

The method is particularly preferably carried out by means of microfluidics, in particular with the aid of single-cell measurements, in particular dynamic single-cell measurements. This enables

a particularly high temporal resolution. Microfluidic methods enable high-resolution functional studies, in particular in combination with optical measuring methods, for example based on fluorescence. Microfluidic methods offer a number of technical advantages, such as miniaturization of bioanalytical methods (Journal of Laboratory Automation, 2013, 18, 350-66), temporally resolved dynamic studies of microorganisms at the single-cell level in a microfluidic cultivation chamber (see examples in the respective review articles, Journal of Molecular Biology, 431, 2019, 4569-4588) or possibilities for screening at high throughput in droplet-based compartments with individual cells or in microcolonies (see examples in the respective review articles) Current Opinion Structural Biology, 2018, 48, 149-156 and Current Opinion in Chemical Biology, 2017, 37, 137-144 and specifically ACS Synthetic Biology, 2017, 6, 1988-1995).

In step a) of the method according to the invention, the quotient of the concentration of the reporter substance in the interior of the cell and the concentration of the reporter substance in the medium surrounding the cell is at least 2 or at most 0.5, preferably at least 5 or at most 0.2, more preferably at least 10 or at most 0.1, more preferably at least 100 or at most 0.01. A difference in the concentration of the reporter substance between the cell interior and the surrounding medium acts as a driving force for a change in the concentration of the reporter substance with increased permeability of the cell membrane. If the concentration in the surrounding medium is higher than in the cell interior, an increase in the concentration in the cell interior results with increased permeability of the cell membrane. However, if the concentration in the surrounding medium is lower than in the cell interior, a reduction in the concentration in the cell interior results with increased permeability of the cell membrane.

According to the invention, the permeability of the cell membrane to the reporter substance is also referred to as permeability. Permeability to a specific molecule is difficult to determine and ultimately depends on a number of physiological factors, such as the intrinsic or passive permeability of the lipid membrane along with active import and export mechanisms. In the context of the present invention, the change in the permeability of the membrane is therefore defined empirically, i.e., preferably in particular specifically by the measured difference of the signal depending on the expression of the membrane protein.

The cell membrane being impermeable to the reporter substance therefore means that the detected signal is constant before inducing the expression of the membrane protein at a constant content of sensor protein in the cell, that is to say, in the case of two or more measurements at different points in time, for example at a distance of one minute, of two minutes, of five 25 minutes or of ten minutes, the quotient of the highest measured signal value and the lowest measured signal value is in a range of 0.75 to 1.25, more

preferably of 0.8 to 1.2, more preferably of 0.85 to 1.15, more preferably of 0.9 to 1.1, more preferably of 0.95 to 1.05.

According to step c) of the method, the expression of a membrane protein that increases the permeability of the cell membrane to the reporter substance is induced. The dynamic range of the method can be adjusted in a targeted manner via the choice of the promoter. In particular in the case of membrane proteins with strong pore-forming properties, it can be advantageous to use a weaker promoter. As a result, a greater scattering can be achieved of the average value with respect to the slope and the half-maximum time, so that the dynamic range of the method can be improved. With weaker protein expression, stronger pore builders can be better differentiated. A particularly preferred weaker promoter is T7.100.sRBS (SEQ ID NO: 27). Promoter T7.wt.sRBS (SEQ ID NO: 26), which results in stronger protein expression, is however also well-suited for carrying out the method according to the invention. The two SEQ ID NOs mentioned show the sequence of the promoter region, including the actual promoter sequence (which is identical for T7.100.sRBS and T7.wt.sRBS) along with the binding site of the lac repressor and the ribosome-binding site (RBS). With respect to the promoter strengths, the two promoter regions differ in the distance between the ribosome-binding site and the binding site for the lac repressor. In the case of the T7.100.sRBS, this distance is shortened by eight nucleotides in the promoter region and thus causes weaker expression of the membrane protein.

In SEQ ID NOs: 50 and 51, the transcription cassettes in the context of pCTRL2 with T7.wt.sRBS promoter (SEQ ID NO:50) or in the context of pCTRL2.T7.100.sRBS with T7.100.sRBS promoter (SEQ ID NO:51) are shown. Three further differences between the two constructs are apparent therein. Firstly, in comparison to T7.wt.sRBS, with T7.100.sRBS, six nucleotides were removed before the actual promoter sequence. Secondly, a different RBS is used. Both the RBS of T7.100.sRBS and the RBS of T7.wt.sRBS are known as strong RBS (Elowitz, M. B., and S. Leibler. 2000. “A Synthetic Oscillatory Network of Transcriptional Regulators.” Nature 403 (6767): 335-38 and Olins, P. O., and S. H. Rangwala. 1989. “A Novel Sequence Element Derived from Bacteriophage T7 mRNA Acts as an Enhancer of Translation of the lacZ Gene in Escherichia Coli.” The Journal of Biological Chemistry 264 (29): 16973-76). Thirdly, in comparison to T7.wt.sRBS, with T7.100.sRBS, four nucleotides were removed after the RBS and replaced by three other nucleotides.

The induction of the expression of a membrane protein according to step c) of the method can take place in particular by addition of an inductor, preferably by addition of isopropyl ß-d-1-thio-galactopyranoside (IPTG), in particular in concentrations of 0.1 mM to 100 mM, for example of 0.2 mM to 50 mM, preferably 0.3 mM to 1 mM or 0.4 mM to 0.6 mM.

In particular in microfluidic embodiments, relevant delays can occur between the addition of the inductor and the time at which the inductor reaches the cell. For example, times of 20 to 30 minutes between the addition of the inductor and the arrival of the inductor in the microfluidic cultivation chamber are not unusual. In particular in such embodiments, the time of inducing expression of the membrane protein according to step c) of the method is thus markedly different from the time of addition of the inductor.

The membrane protein increasing the permeability of the cell membrane to the reporter substance accordingly means that the signal value determined according to step d), in comparison to the comparative value determined according to step b), reaches higher values before the induction of the expression of the membrane protein, in particular such that the quotient of a signal value according to step d) and a signal value according to step b) is preferably in a range of >1.25 to 10, more preferably of 1.5 to 7.5, more preferably of 2 to 5. Step d) is preferably carried out at least twice, more preferably at least three times, more preferably at least five times, more preferably at least ten times, more preferably at least twenty times, more preferably at least fifty times, more preferably at least a hundred times at different points in time, in particular regularly, for example at a distance of 1 to 10 minutes or 2 to 5 minutes, in particular at a distance of one minute, of two minutes, of five minutes or of ten minutes. In this way, a temporal profile can be created, which is particularly helpful when evaluating the properties of the proteins investigated. Step d) is preferably carried out not more than a thousand times.

According to step e) of the method according to the invention, one or more comparative parameters are calculated from the signal strengths detected in steps b) and d). Such a comparative parameter is particularly helpful in order to compare the effect of two or more different membrane proteins on the permeability of the cell membrane to the reporter substance with one another, wherein a comparative parameter is determined for each of the membrane proteins with the method of the invention. A comparative parameter can also be used for comparing two or more different sensor proteins.

A comparative parameter can be calculated particularly easily, for example by forming a difference or a quotient of the signal strengths detected in steps b) and d).

The quotient of a signal value according to step d) and a signal value according to step b) have already been described above. The difference of a signal value according to step d) and a signal value according to step b) can also be formed accordingly and serve as comparative parameter. Such simple comparative parameters are advantageous in particular when step d) was carried out only once, in particular in the case of a determination at a point in time at which a relevant change in the signal strength no longer results, preferably when step d) is carried out 60 to 240 minutes, for example 90 to 150 minutes, after step c).

According to the invention, it is also possible to normalize the signal strengths, for example in the context of the calculation of the comparative parameter. Normalization can, for example, be performed to the optical density at 600 nm (OD600 nm). OD600 nm is a measure of the number of cells in a sample. However, normalization to OD600 nm entails various disadvantages. For example, such a normalization can change the kinetics of the curve. Furthermore, such a normalization is problematic for measurements in which OD600 nm exceeds a value of 1. Normalization to OD600 nm is also not advantageous for cell-lyzing variants, since OD600 nm decreases over time in such variants and can falsify the signal. Normalization can therefore preferably take place according to the invention in different ways, in particular via negative controls. The selection of the negative control depends on the reporter substance that is to be the focus. In particular, the negative control must be different from the reporter substance. For example, normalization can take 10 μlace with the aid of H⁺ or K⁺ as the negative control if H⁺ or K⁺ are not the reporter substance, for example with Ca²⁺ or glutamate as the reporter substance. For these negative controls, in particular the BM2 proton channel or a K⁺-specific channel are relevant.

In many embodiments of the invention, normalization is dispensed with. For example, when carrying out the method in microtiter plates, normalization is preferably dispensed with, since in the case of microtiter plates, the signal strength is already averaged over many cells anyway, so that normalization is generally not associated with further advantages. Even if the time profile of the signal strength is the focus (e.g., with the aid of the half-maximum time (T1/2) and/or the slope of the signal strength as a function of the time, see equation 1), the properties to be investigated can be imaged excellently without normalization entailing further advantages. The kinetics in some embodiments of the invention are independent of the expression pattern. This means that, for example in the case of different expression of the sensor protein, only a change in the maximum signal strength can occur, but that T1/2 and the slope

remain the same. Even if pores are used as membrane proteins in order to increase the permeability of the cell membrane to the reporter substance, normalization generally does not entail any advantages, since the expression level of the sensor protein is so high that a maximum signal strength is achieved anyway.

However, in embodiments in which the half-maximum time and/or the slope of the signal strength depend on the expression level of the membrane protein, normalization can be carried out. Normalization can be achieved, for example, by using one or more membrane proteins with respectively known parameters for the half-maximum time and/or for the slope as control references.

However, in embodiments in which the half-maximum time and/or the slope of the signal strength depend on the expression level of the membrane protein, normalization can be dispensed with by taking care that the expression level of the membrane proteins to be investigated does not differ or only differs insignificantly. This can be achieved, for example, by testing under comparable conditions, in particular by expressing the membrane proteins with the aid of the same promoter or the same promoter region.

A comparative parameter calculated from the signal strengths can in particular be a kinetic parameter, i.e., a parameter by means of which the time profile of the change in the signal strength can be characterized. As described above, step d) is preferably carried out several times at different points in time, so that a temporal profile of the change in the signal strength can be created. A temporal profile of the signal strength with a sigmoidal curve profile regularly results. The time profile of the signal strength can thus preferably be described with a sigmoid function. The calculation of the comparative parameter from the signal strengths according to step e) preferably includes empirically fitting the data obtained on the signal strengths with a sigmoid function, wherein the comparative parameter calculated from the signal strengths is the half-maximum time c and/or the maximum slope d.

The person skilled in the art knows many equations with a sigmoidal curve profile, which are in principle suitable for describing the temporal signal strength profile. Such equations describe the sigmoidal curve profile preferably with the aid of the following four parameters: baseline, saturation, maximum slope and half-maximum time.

The data obtained are particularly preferably fitted empirically with the following equation 1. The half-maximum time c and the slope d can thereby be determined quantitatively.

$$f ( x ) = a + \frac{b}{1 + {e}^{\frac{- ( x - c )}{d}}} \qquad Equation 1$$

In equation 1, f(x) describes the signal strength f determined at time x. Here, x=0 corresponds to the time of induction of expression. The parameter a describes the lower signal level (baseline) and the parameter b describes the upper signal level (saturation). The parameter c indicates the half-maximum time, i.e., the time x at which the signal strength f has reached half of its maximum value fₘₐₓ. The half-maximum time c corresponds in particular to the time at the inflection point of the sigmoidal curve. The parameter d indicates the maximum slope of f(x), i.e., the maximum rate of change of the signal strength as a function of time. The maximum slope d is present at the inflection point of the sigmoidal curve. A comparative parameter calculated from the signal strengths is particularly preferably the half-maximum time c and/or the maximum slope d. Equation 1 requires that a high value for d corresponds to a low slope, and that, conversely, a low value for d corresponds to a high slope.

With the method according to the invention, it is possible to compare the properties of various membrane proteins to one another. The properties of various sensor proteins can also be compared to one another. For this purpose, the comparative parameters, obtained according to step e), of the various proteins investigated are preferably compared to one another. It is preferable to draw conclusions regarding the properties of the membrane proteins and/or sensor protein from the comparative parameter according to step e) of the method according to the inventions. A particularly large (high maximum value fₘₐₓ) and/or rapid (low half-maximum time c and/or high slope, which appears as a low value d) increase in the signal strength as a result of the induction of the expression of the membrane protein preferably indicates that the protein is a particularly advantageous protein. A particularly small (low maximum value fₘₐₓ) and/or slow (high half-maximum time c and/or low slope, which appears as a high value d) increase in the signal strength on the other hand indicates that the protein is a particularly disadvantageous protein. Ultimately, however, it depends on the application scenario. If a rapid and strong inflow of the reporter substance is desired, the slope d should be high and the half-maximum time c low. If, on the other hand, a slow inflow of the reporter substance is desired, a small slope d and a high half-maximum time care preferred.

When the results of different membrane proteins are compared to one another, the proteins investigated can be classified based on the comparison results. A particularly large and/or rapid increase in the signal strength as a result of the induction of the expression of the membrane protein shows that the latter increases the permeability of the cell membrane to the reporter substance to a particular degree. On the other hand, a particularly small and/or slow increase in the signal strength shows that the permeability of the cell membrane to the reporter substance was increased only to a small extent. In the extreme case, there is no increase in the permeability at all, so that there is no increase in the signal strength. Depending on the desired permeability properties, the appropriate membrane proteins can then be selected based on the results obtained. In order to obtain comparable results, it is advantageous to use the same sensor proteins in all experiments in the case of an investigation of various membrane proteins.

However, the method according to the invention is also suitable for characterizing various sensor proteins. In this case, it is advantageous to use the same membrane proteins in all experiments, in order to be able to ascribe differences in the increase in the signal strength solely to differences between the tested sensor proteins. A particularly large increase in signal strength (high maximum value fₘₐₓ) as a result of the induction of the expression of the membrane protein in this case shows that the sensor protein achieves a particularly high signal strength per reporter substance. A particularly rapid increase in the signal strength (low half-maximum time c and/or high slope d) shows a high temporal resolution of the sensor protein. A particularly small (low maximum value fₘₐₓ) and/or slow (high half-maximum time c and/or low slope d) increase in the signal strength on the other hand shows that the signal strength per reporter substance or the temporal resolution of the sensor protein is not high. In the extreme case, no increase in the signal strength takes place at all. This shows that the tested sensor protein is not suitable for detecting the corresponding reporter substance at all. Depending on the desired sensor properties, the appropriate sensor proteins can be selected based on the results obtained.

For a selection of suitable proteins based on the comparison results discussed above, it is advantageous if the proteins are easily identifiable. The screening method therefore preferably comprises the further step of:

f) determining the DNA sequence encoding the membrane protein and/or the DNA sequence encoding the sensor protein.

Alternatively or additionally, the protein sequence of the membrane protein and/or of the sensor protein can also be determined.

The DNA sequence can be determined with significantly simpler means than the protein sequence. The DNA sequence is therefore preferably determined. The DNA sequence can be determined using DNA sequencing methods. A variety of such sequencing methods are known to the person skilled in the art.

In turn, the protein sequence can be easily determined from the DNA sequence on the basis of the known triplet coding.

According to step a) of the method of the present invention, cells and a medium surrounding the cells are provided. The cells are preferably microorganisms, in particular selected from the group consisting of bacteria, unicellular fungi, microscopic algae, protozoa and combinations thereof. Microorganisms are preferably used within the scope of the present invention due to their comparatively high transformation efficiency and “genetic formability.”

The cells can, for example, be gram-negative or gram-positive bacteria or yeasts. The cells are particularly preferably selected from the group consisting of E. coli, S. cerevisiae, B. subtilis and combinations thereof. The cells are particularly preferably selected from the group consisting of E. coli, B. subtilis and combinations thereof.

The cells are particularly preferably prokaryotic microorganisms, in particular bacteria, for example selected from the group consisting of gram-positive bacteria and gram-negative bacteria. Even more preferably, the cells are gram-positive bacteria, in particular Bacillus subtilis.

Particularly preferably, the cell is E. coli. Very particularly preferably, the cell is E. coli and the cell membrane is the inner membrane of E. coli.

With the method according to the invention, it is possible to compare the properties of various membrane proteins with one another. Preferably, steps a) to e), more preferably a) to f), are carried out for at least 5, preferably at least 10, more preferably at least 20, more preferably at least 50, more preferably 10² to 10⁸ membrane proteins with different amino acid sequences.

With the method according to the invention, it is possible to compare the properties of various sensor proteins with one another. Preferably, steps a) to e), more preferably a) to f), are carried out for at least 5, preferably at least 10, more preferably at least 20, more preferably at least 50, more preferably 10² to 10⁸ sensor proteins with different amino acid sequences.

The method of the present invention includes detecting a signal that depends on the concentration of a reporter substance in the cell and is generated with the aid of a sensor protein that is expressed in the cell.

The signal is preferably a fluorescence signal, a bioluminescence signal or an absorption signal.

The sensor protein is preferably selected from the group consisting of cation-specific protein sensors, amino acid-specific protein sensors and combinations thereof, in particular from the group consisting of Ca²⁺-specific protein sensors, L-Glu-specific protein sensors and combinations thereof. Particularly preferably, the sensor protein is selected from the group consisting of R-GECO-1 (SEQ ID NO:23), G-GECO-1 (SEQ ID NO:24), iGluSnFR (SEQ ID NO:25) and variants of said sensor proteins, in particular variants that have sequence identity to the protein sequence of at least one of the sensor proteins R-GECO-1, G-GECO-1 and iGluSnFR of at least 90%, more preferably at least 95%, more preferably at least 98%, more preferably at least 99%. The method of the present invention is in particular suitable for comparing various variants of a sensor protein with one another and for selecting those with desired properties from the plurality of variants. Relevant properties for the selection of certain sensor proteins can, for example, be the signal strength per reporter substance and/or the temporal resolution of the sensor protein. It is frequently desirable to select sensor proteins with particularly high sensitivity and/or specificity with regard to the respective reporter substance. The person skilled in the art knows what sequence identity between two protein sequences means and how it can be determined. If a certain sequence is to be compared to a sequence listed in the sequence listing of the present invention, one of the known sequence comparison algorithms can in particular be used, for example BLAST. A comparison of the two sequences enables a simple determination of the number of amino acids that occur in the two sequences in the identical manner. The term “sequence identity” denotes the ratio of the number of these identical amino acids to the total number of amino acids of the sequence listed in the sequence listing of the present invention.

The reporter substance is preferably selected from the group consisting of cations, amino acids, peptides, proteins and metabolic products and metabolic intermediates (wherein the metabolic products and metabolic intermediates in particular comprise aromatic substances, such as 3,4-hydrobenzoic acid, 4-hydroxybenzoic acid, 1,2-catechol and cis,cis-muconic acid) and combinations thereof. Particularly preferably, the reporter substance is selected from the group consisting of cations, amino acids and combinations thereof, in particular from the group consisting of Ca²⁺, glutamate and combinations thereof.

The reporter substance can be a cation. Various cations are suitable as reporter substances. The reporter substance is preferably selected from the group consisting of Ca²⁺, Na⁺, K⁺, Mg²⁺, Zn²⁺ and combinations thereof. Particularly preferably, the reporter substance is Ca²⁺.

The reporter substance can be an amino acid. Various amino acids are suitable as reporter substances. The amino acid is preferably selected from the group consisting of glutamate (L-Glu), glycine (L-Gly), threonine (L-Thr) and combinations thereof. A particularly preferred amino acid is glutamate (L-Glu).

With the method according to the invention, it is possible to compare the properties of various membrane proteins with one another. The membrane protein is preferably selected from the group consisting of pore-forming membrane proteins, membrane transporters and combinations thereof, in particular from the group consisting of pinholins (in particular S²¹-68, S²¹-71, S²¹-71 M4A, ^{SGS}ΔTMD1-S²¹68, ^{TVMV}ΔTMD1-S²¹68), holins (S¹⁰⁵, S¹⁰⁷, S¹⁰⁷-M3A, T4, T4^{ΔC-Tail}, ^{ΔN-Tail}T4^{Δc-Tail}), viral potassium channels (in particular K_{CV}^{NTS}, K_{CV}^{NTS G77S}, K_{CV}^{PBCV1}), components of the toxin/antitoxin (TA) system (in particular HokB, TisB), hemolysin proteins (in particular αHLA), artificial pores (in particular cWZA), proton porters (in particular BM2), hCV membrane proteins (in particular HCVTME1, HCV TME2) and combinations thereof, particularly preferably from the group consisting of holins, pinholins, ion channels and combinations thereof. Very particularly preferably, the membrane protein is selected from the group consisting of S²¹-68 (SEQ ID NO:1), S²¹-71 (SEQ ID NO:2), S²¹-71 M4A (SEQ ID NO:3), ^{SGS}ΔTMD1-S²¹68 (SEQ ID NO:4), ^{TVMV}ΔTMD1-S²¹68 (SEQ ID NO:5), S¹⁰⁵ (SEQ ID NO:6), S¹⁰⁷ (SEQ ID NO:7), S¹⁰⁷-M3A (SEQ ID NO:8), T4 (SEQ ID NO:9), T4^{ΔC_Tail} (SEQ ID NO:10), ^{ΔN-Tail}T4^{ΔC-Tail} (SEQ ID NO:11) K_{CV}^{NTS} (SEQ ID NO:12) K_{CV}^{NTS G77S} (SEQ ID NO:13) K_{CV}^{PBCV1} (SEQ ID NO:14), HokB (SEQ ID NO:15), TisB (SEQ ID NO:16), αHLA (SEQ ID NO:17), cWZA (SEQ ID NO:18), BM2 (SEQ ID NO:19), HCVTME1 (SEQ ID NO:20), HCV TME2 (SEQ ID N0:21) and variants of said proteins, in particular variants that have sequence identity to at least one of the sequences of SEQ ID NO: 1-21 of at least 90%, more preferably at least 95%, more preferably at least 98%, more preferably at least 99%. The method of the present invention is in particular suitable for comparing various variants of a membrane protein with one another and for selecting those with desired properties from the plurality of variants. A particularly relevant property for the selection of certain membrane proteins can, for example, be the extent of permeability to a reporter substance. It is frequently desirable to select membrane proteins with particularly high sensitivity and/or specificity with regard to the respective reporter substance.

Further, preferred membrane proteins are S²¹-66 (SEQ ID NO:28), S²¹-64 (SEQ ID NO:29), S²¹-62 (SEQ ID NQ:30), S²¹-60 (SEQ ID NO:31), S²¹-58 (SEQ ID NO:32), S²¹-56 (SEQ ID NO:33), S²¹-54 (SEQ ID NO:34), S²¹-52 (SEQ ID NO:35), S²¹-50 (SEQ ID NO:36), S²¹-48 (SEQ ID NO:37), S²¹-46 (SEQ ID NO:38), S²¹-44 (SEQ ID NO:39), S²¹-42 (SEQ ID NQ:40), S²¹-40 (SEQ ID NO:41), S²¹-38 (SEQ ID NO:42) and variants of said proteins, in particular variants that have sequence identity to at least one of the sequences of SEQ ID NO: 28-42 of at least 90%, more preferably at least 95%, more preferably at least 98%, more preferably at least 99%. Particularly preferred membrane proteins are S²¹-64 (SEQ ID NO:29), S²¹-62 (SEQ ID NQ:30), S²¹-60 (SEQ ID NO:31), S²¹-58 (SEQ ID NO:32), S²¹-56 (SEQ ID NO:33), S²¹-54 (SEQ ID NO:34), S²¹-52 (SEQ ID NO:35), S²¹-50 (SEQ ID NO:36), S²¹-48 (SEQ ID NO:37) and variants of said proteins, in particular variants that have sequence identity to at least one of the sequences of SEQ ID NO: 29-37 of at least 90%, more preferably at least 95%, more preferably at least 98%, more preferably at least 99%. Very particular preference is given to the pinholin variant S²¹-48 (SEQ ID NO:37) and variants thereof, in particular variants that have sequence identity to SEQ ID NO:37 of at least 90%, more preferably at least 95%, more preferably at least 98%, more preferably at least 99%.

Further, preferred membrane proteins are N-terminal variants of S²¹-71 according to SEQ ID NO:43 and variants thereof, in particular variants that have sequence identity to SEQ ID NO:43 of at least 90%, more preferably at least 95%, more preferably at least 98%, more preferably at least 99%. In SEQ ID NO:43, Ala, Arg, Asn, Asp, Gin, Glu, Gly, His, lie, Leu, Lys, Phe, Ser, Thr, Trp, Tyr or Val can be provided independently of one another at any of the positions indicated by “Xaa.”

Particularly preferred membrane proteins are the N-terminal variants of ²¹-71 according to SEQ ID NO:44 (clone P14E5), SEQ ID NO:45 (clone P14C11), SEQ ID NO:46 (clone P1503), SEQ ID NO:47 (done P7D10), SEQ ID NO:48 (clone P3G3), SEQ ID NO:49 (clone P3E5) and variants of said proteins, in particular variants that have a sequence identity to at least one of the sequences of SEQ ID NO: 44-49 of at least 90%, more preferably at least 95%, more preferably at least 98%, more preferably at least 99%.

In comparison to known methods, the screening method of the present invention is characterized in particular by its high temporal resolution capability. The temporal resolution capability is determined in particular by the speed of expression and the speed of the membrane protein when increasing the permeability of the cell membrane to the reporter substance. In addition, there are further factors, for example the sensitivity of the method of detecting the signal. A particularly high temporal resolution can be achieved, for example, with the aid of dynamic single-cell measurements by means of microfluidics.

Step d) is preferably carried out at most 120 minutes, preferably at most 60 minutes, more preferably at most 45 minutes, more preferably at most 30 minutes, more preferably at most 15 minutes, more preferably at most 10 minutes after step c). However, keeping a certain time distance between steps c) and d), for example at least 2 minutes or at least 5 minutes, can be advantageous, in particular in order to ensure particularly robust signal detection.

The time between steps c) and d) can be flexibly adapted to the kinetics to be investigated in each case. In embodiments of the invention, the detection according to step d) takes place in a time period of 2 to 1000 minutes, for example 5 to 750 minutes, 10 to 500 minutes, 15 to 400 minutes, 20 to 300 minutes, 30 to 240 minutes, 45 to 180 minutes or 60 to 150 minutes after induction according to step c) of the method. Step d) is preferably carried out at most 1000 minutes, preferably at most 750 minutes, more preferably at most 500 minutes, more preferably at most 400 minutes, more preferably at most 300 minutes, more preferably at most 240 minutes after step c).

The present invention also relates to a kit for carrying out the screening method. Preferably, the kit comprises

i. DNA encoding at least two membrane proteins and DNA encoding a sensor protein, or
ii. DNA encoding at least two sensor proteins and DNA encoding a membrane protein.

In case i., the kit preferably contains DNA encoding at least 5, preferably at least 10, more preferably at least 20, more preferably at least 50, more preferably 10² to 10⁸ membrane proteins. In case ii., the kit preferably contains DNA encoding at least 5, preferably at least 10, more preferably at least 20, more preferably at least 50, more preferably 10² to 10⁸ sensor proteins. The DNA for the various membrane proteins (case i.) or sensor proteins (case ii.) are preferably present in a separate form in the kit. This makes it easier to carry out the method in a targeted manner with the desired proteins.

The DNA is preferably present in the form of plasmid DNA.

The DNA can be present in isolated form in the kit, for example in dried form or in the form of solutions. The DNA can also be located within cells. In this case, the cells are also part of the kit.

The kit can also comprise the reporter substance, for example in the form of a salt.

### BRIEF DESCRIPTION OF THE FIGURES

FIG. 1: Forming or opening a (protein) nanopore with promiscuous or 30 specifically Ca2+-specific permeability, for example in the inner membrane of Escherichia coli, results in inflow or outflow of Ca2+. The possibility can consequently be used to measure the ligand-dependent fluorescence of a genetically encoded sensor in the cell interior at the single-cell level. In practice, this enables the construction of pore-forming membrane peptides by means of various screening formats of microtiter plates up to the single-cell level by means of a flow cytometer.

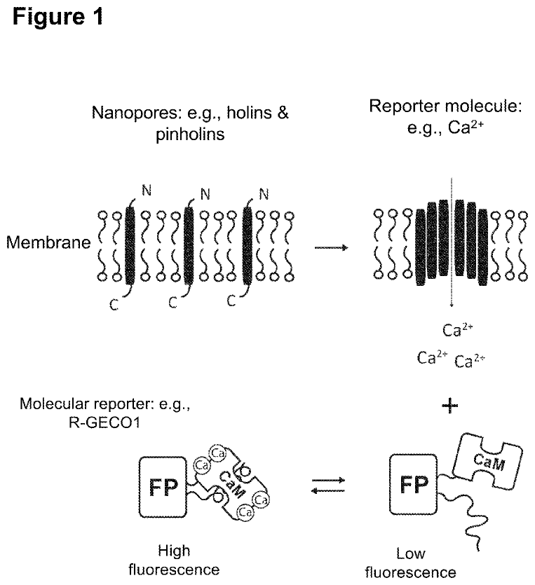

FIG. 2: Forming or opening a (protein) nanopore with promiscuous permeability, for example in the inner membrane of Escherichia coli, results in inflow or outflow of a ligand, such as a specific metabolic product. The possibility can consequently be used to measure the ligand-dependent fluorescence of a genetically encoded sensor in the cell interior at the single-cell level. In practice, this enables the construction of customized protein sensors by means of various screening formats up to the single-cell level by means of a flow cytometer.

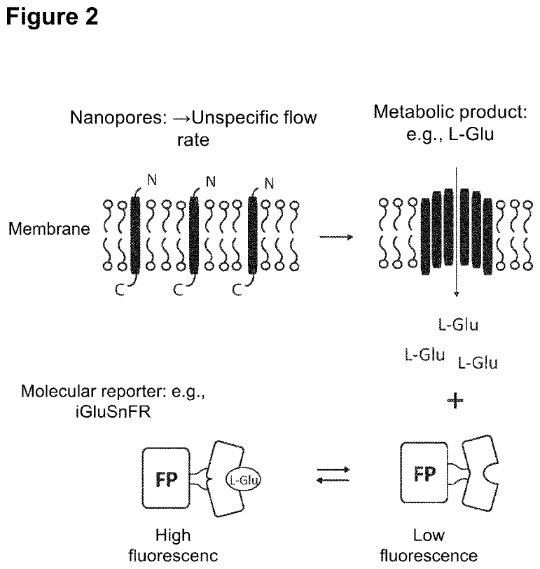

FIG. 3: Assay validation with (A) pACYCT2-^{TVMV}ΔTMD1-S²¹68 (negative control) and (B) pACYCT2-^{SGS}ΔTMD1-S²¹68 (positive control), in double transformation with pPRO24-R-GECO1 in the microtiter plate test in each case. While the pACYCT2-^{TVMV}ΔTMD1-S²¹68 variant after induction shows a continuous OD_{600 nm} increase and thus unchanged growth, the growth curve in the case of the pACYCT2-ATMD1-S²¹68 variant stagnates promptly. In correlation thereto, the fluorescence channel (Ex: 555 nm; Em: 600 nm) shows a sigmoidal increase of the R-GECO1 signal in the case of the toxic ^{SGS}ΔTMD1-S²¹68 variant, while the negative control ^{TVMV}ΔTMD1-S²¹68 shows a continuous baseline; The dashed line shows induction by 0.5 mM IPTG at 120 min (SD, n=3).

FIG. 4: Analysis of the pore-forming properties for the wild-type holins or pinholins S²¹-68 and S¹⁰⁵ along with the associated anti-holins/pinholins.

FIG. 5: Analysis of the pore-forming properties for the wild-type T4 holin and holin fragments derived therefrom with deleted N- and C-terminal domains.

FIG. 6: Analysis of the pore-forming properties for different variants of K_{CV} channels with different opening states.

FIG. 7: Analysis of the pore-forming properties for different nanopores HokB, TisB, cWZA along with a-hemolysin.

FIG. 8: Analysis of the pore-forming properties for different nanopores and transmembrane peptides.

FIG. 9: Model selection for the 1:5 dilution of pACYCT2-^{SGS}ΔTMD1-S²¹68 to pACYCT2-^{TVMV}ΔTMD1-S²¹68 (20% positive control and 80% negative control)+pPRO24-R-GECO1. (A) Blank H6, positive control H7-H9; negative control H10-H12. The values of A (before induction) and B (after induction) are RFU (Ex: 555 nm; Em: 600 nm) divided by the OD_{600 nm}; C shows the ratio of B to A. (B) After induction of R-GECO1, but before induction of the pore, the signal is continuously low. If the pore is induced, the fluorescence of individual cavities that contain the positive control pACYCT2-ΔTMD1-S²¹68 increases. (C) For better clarity, the ratio of B to A can be represented and the threshold value can be determined. In A and B, the blank in H6 shows a positive signal, since the autofluorescence of the medium divided by a very low OD_{600 nm} (since there are no bacteria present therein) results in an extremely high signal.

FIG. 10: Model selection for the 1:10 dilution of pACYCT2-ΔTMD1-S²¹68 to pACYCT2-^{TVMV}ΔTMD1-S²¹68 (20% positive control and 80% negative control)+pPRO24-R-GECO1. (A) Blank H6, positive control H7-H9; negative control H10-H12. The values of A (before induction) and B (after induction) are A.U. (Ex: 555 nm; Em: 600 nm) divided by the OD_{600 nm}; C shows the ratio of B to A. Similarly to the 1:5 model selection, the signal after induction of R-GECO1, but before induction of the pore is low. (B) After induction of the pore, the signal (Ex: 555 nm, Em: 600 nm) also strongly increases in individual cavities here. (C) If the ratio of the data after the induction and the data before the induction of the pore is formed, the signal-to-noise ratio can be improved once again. In A and B, the blank in H6 shows a positive signal, since the autofluorescence of the medium divided by a very low OD_{600 nm} (since there are no bacteria present therein) results in an extremely high signal.

FIG. 11: Overview of pore-forming properties from the ^{3×NNK}S²¹68 library measured at half-maximum time. Holins and pinholin fragment are each included as a reference.

FIG. 12: Overview of pore-forming properties from the ^{3×NNK}S²¹68 library measured at the slope. Holins and pinholin fragment are each included as a reference.

FIG. 13: Characterization of five individual variants that were selected from the ^{3×NNK}S²¹68 library. The selected variants in this case each have, as a function of N-terminal mutations, different inclinations to form pores.

FIG. 14: After expression of various pore-forming membrane peptides, the outflow of the GECO from the cell was determined quantitatively by means of a fluorescent SDS-PAGE in the cell pellet and in the supernatant (the latter is assessed as loss of cellular integrity). In this case, the GECO and the various nanopores were expressed as indicated for different times. In contrast to ^{SGS}ΔTMD1-S²¹68 and BM2, the expression of the T4 holin and HokB results in complete lysis (→R-GECO 2 h//Pore 3h) in the exponential growth phase. The anti-holins S²¹-71 and S¹⁰⁷ along with TisB result in averaged values. In the case of the anti-holins S²¹-71 and S¹⁰⁷, it should however be considered that the pore-forming holins can also be expressed by shifting the translation grid and can thus result in a higher-than-average lysis of the cell.

FIG. 15: Temporally resolved measurement of the GECO-related fluorescence in the pellet in comparison to the supernatant as a function of cell growth measured using OD600. For ^{SGS}ΔTMD1-S²¹68 and the BM2 proton channel, the fluorescence is retained at stagnant growth. In contrast, expression of HokB and the T4 holins results in a loss of cellular integrity and a time-dependent increase in fluorescence in the supernatant.

FIG. 16: Assay validation with the pinholin fragments in double transformation with pPRO24-R-GECO1. Recording of the fluorescence of colonies at 520 nm for 0.3 s. p<0.0001. N=2, n>180.

FIG. 17: Example image for the model selection of pACYCT2-^{SGS}ATMD1-S68 to pACYCT2-^{TVMV}ATMD1-S²¹68 (20% positive control and 80% negative control)+pPRO24-R-GECO1. The colonies of the positive control in each case appear dark to black in contrast to colonies of the negative control, which appear light-gray.

FIG. 18: Exemplary FACS cytograms. All Events (left diagrams): Relates to forward and backward scatter and provides an indication of cellular integrity (preserved independently of the expression of ^{SGS}ΔTMD1-S²¹68 and ^{TVMV}ΔTMD1-S²¹68 in each case); M (right diagrams): Relates to fluorescence as a function of the expression of ^{SGS}ΔTMD1-S²¹68 and ^{TVMV}ΔTMD1-S²168 in each case (additionally separated after the forward scatter): Two different populations are evident as a function of the expression of the two different membrane peptides; M (bottom diagrams): Relates exclusively to the GECO-related fluorescence signal. Two different populations are again evident as a function of the expression of the two different membrane peptides.

FIG. 19: Exemplary FACS cytograms. All Events (top left diagram): Relates to forward and backward scatter and provides an indication of cellular integrity (preserved independently of the expression of ^{SGS}ΔTMD1-S²¹68 and ^{TVMV}ΔTMD1-S²¹68 in each case); M: Relates to the distribution of the fluorescence signal (as a function of the forward scatter). The marked gate (circled) marks the region that is preselected; Fluorescence All Events (top right diagram): Relates to the distribution of the fluorescence signal within the preselected gate (independence of the forward scatter this time): U: Relates exclusively to the distribution of the fluorescent signal in the preselected gate, in which the Events with the highest fluorescence (in this case 17.1%) are ultimately selected. 1: DNA conductor; 2: PCR fragment ^{SGS}ΔTMD1-S²¹68; 3: PCR fragment ^{TVMV}ΔTMD1-S²¹68; 4: Preselection PCR; 5: Post-selection PCR.

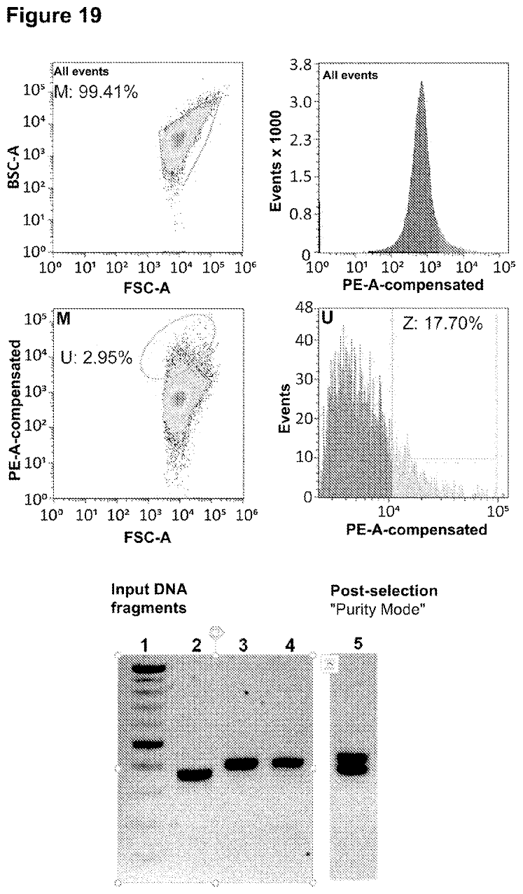

FIG. 20: Expression of iGluSNFr results in an increase in fluorescence, which, as a result of co-expression of the pore-forming peptide ^{SGS}ΔTMD1-S²¹68, causes an outflow of L-Glu from the cell and thus a reduction in the fluorescence. No reduction in fluorescence is observed upon co-expression of the BM2 proton channel and of the non-pore-forming membrane peptide ^{TVMV}ΔTMD1-S²¹68.

FIG. 21: Expression of iGluSNFr results in an increase in fluorescence, which, as a result of co-expression of the pore-forming peptides S²¹-71-M4A, S107-M3A and HokB, causes an outflow of L-Glu from the cell and thus a reduction in the fluorescence.

FIG. 22: Expression of iGluSNFr results in an increase in fluorescence. Co-expression of different K⁺-conducting ion channels however does not cause any outflow of L-Glu and thus does not cause any reduction in the fluorescence.

FIG. 23: Exemplary image details of the fluorescence micrographs of cells in the microfluidic cultivation chamber, shown for the S²¹68 pinholin at four different times, after 21 min (0:21 h), 63 min (1:03 h), 105 min (1:45 h), and 399 min (6:39 h).

FIG. 24: Summary of the fluorescent signal curve for the S²¹68 pinholin and for the T4 holin in a microfluidic cultivation chamber. For the analysis, all cells from a single cultivation chamber, the fluorescence in relative fluorescence units (RFU) was plotted against the time. The induction of the nanopore is shown with a dashed line.

FIG. 25: Comparison of the ^{3×NNK}S21⁶⁸ library, respectively investigated in the context of the two different expression vectors pCTRL2 and pCTRL2.T7.100.sRBS. In the case of the more weakly expressing promoter pCTRL2.T7.100.sRBS, the scattering of the half-maximum time c and of the slope d is greater in comparison to the pCTRL2 in the context of the T7.wt.sRBS promoter. Using a more weakly expressing promoter makes it possible, by way of example, in the context of a library selection, to better differentiate the pore-forming properties.

FIG. 26A: Summary of the fluorescent signal curve for the ^{3×NK}S²¹68 library in microtiter plates. For the analysis, for each individual amino acid at the three different positions X₁, X₂ and X₃ (marked in the graph in each case as position 1, position 2, and position 3) in order to calculate in each case the average half-maximum time c and the average slope d. The average values were calculated independently of the context of the sequence.

FIG. 26B: Summary of the fluorescent signal curve for the ^{3×NNK}S²¹68 library in microtiter plates. For the analysis, for the individual amino acids at the three different positions X₁, X₂ and X₃ (marked in the graph in each case as position 1, position 2, and position 3), groups with comparable chemical properties were created in order to calculate in each case the average half-maximum time c and the average slope d. The average values were calculated independently of the context of the sequence.

FIG. 27: Summary of the fluorescent signal curve for the S²¹68 truncation library in microtiter plates. For the analysis, the kinetic profile curves for the individual truncation variants were fitted empirically with equation 1, in order to determine the half-maximum time c and the slope d quantitatively.

FIG. 28: Summary of the fluorescent signal curve for the S²¹68 truncation library in colonies on agar plates. For the analysis, the data from a constant image section of the agar plate were analyzed with the aid of image analysis software and the maximum fluorescence was plotted against the time. The numbers along the X-axis indicate the truncation variant in each case.

### EXAMPLES

The present invention is to be explained in more detail by the following examples.

### Overview of Expression Constructs and Screening Strains for Nanopores and Ion Channels

### Protein of Expression Constructs:

pPRO24: The pPRO24 plasmid contains a class-A on and a titrable propionate-inducible promoter (Environmental Microbiology 2005, 71, 6856-62). This enables flexible control of the expression of a molecular sensor, such as the Ca²⁺-specific R- and G-GECO (Science, 2011, 333, 1888-1891).

pOSIP-CH: The pOSIP-CH enables the incorporation of gene expression cassettes of co-expression into defined sites of the E. coli genome. Specifically, the incorporation is made possible by co-expression of the HK022 integrase. 25 Integration of gene expression cassette, e.g., for the R and G-GECO sensors, allows more stable propagation and expression of the R- and G-GECO cassettes in E. coli (caused by uniform copy numbers).

pACYCT2: The pACYCT2 plasmid contains a class-B on (p15A)+chloramphenicol resistance and a tac promoter induced by IPTG (Nucleic Acids Research, 2013, 41, e150). Used as expression vector for different pinholins and variants, derived therefrom, of the S²¹-68 pinholins: In particular, the pore-forming and non-pore-forming membrane peptides ^{SGS}ΔTMD1-S²¹68 (PNAS, 2009, 106, 18966-18971) and ^{TVMV}ΔTMD1-S²¹-68.

pTeT7W: Dual promoter vector based on the pACYCT2 backbone with p15A ori+chloramphenicol resistance and lacI repressor for independent co-expression of two proteins. In addition to the given elements of the pACYCT2, a TetR expression cassette together with promoter from the pASK and the tac was replaced with the T7 promoter from the pET24. This allows the expression of the two proteins by means of IPTG and tetracycline.

pCTRL2: Expression vector for the expression of toxic membrane channels and membrane peptides. Based on the pACYCT2 vector. The gene expression cassette is flanked on both sides by transcription terminators (ACS Synthetic Biology, 2015, 20, 4, 265-73) and the basal expression of potentially non-specific proteins is to be suppressed. The expression is carried out under control of a lacI repressor mutant with increased suppression of gene expression (Microbial Cell Factories, 2013, 12, 67). The underlying promoter is denoted in the present disclosure by T7.wt.sRBS in each case. The transcription cassette in the context of pCTRL2 with T7.wt.sRBS promoter is shown in SEQ ID NO:50 by way of example for the expression of the fluorescent protein mkO-kappa. The fluorescent protein mkO-kappa is in each case replaced by a membrane protein in the individual experiments.

pCTRL2.T7.100.sRBS: Expression vector for the expression of toxic membrane channels and membrane peptides. Based on the pACYCT2 vector. The gene expression cassette is flanked on both sides by transcription terminators (ACS Synthetic Biology, 2015, 20, 4, 265-73) to support the basal expression of potentially non-specific proteins. The expression is carried out under control of a lacI repressor mutant with increased suppression of gene expression (Microbial Cell Factories, 2013, 12, 67). In comparison to pCTRL2, the distance in pCTRL2.T7.100.sRBS between the ribosome-binding site and the binding site for the lac repressor is shortened in this expression vector and thus causes a lower expression of the respective nanopore. The underlying promoter is denoted in the present disclosure by T7.100.sRBS in each case. The transcription cassette in the context of pCTRL2.T7.100.sRBS with T7.100.sRBS promoter is shown in SEQ ID NO:51 by way of example for the expression of the fluorescent protein mkO-kappa. The fluorescent protein mkO-kappa is in each case replaced by a membrane protein in the individual experiments.

E. coli Strains for Assay Development and Screening:

BL21(DE3) with pPRO24R-GECO1. The BL21(DE3) E. coli were made chemically competent together with pPRO24-R-GECO1 or pPRO24-G-GECO1. This strain enables the efficient transformation and screening of pore-forming membrane peptides, nanopores and ions channels that are expressed via pACYCT2, pTeT7W or pCTRL2.

BL21(DE3) with genomically Integrated R- and G-GECO1: This strain contains the expression cassette for R- or G-GECO1 in a genomically integrated variant. Specifically, the gene for R- or G-GECO1 was stably integrated by means of the pOSIP-CH plasmid into the attB site of the E. coli genome (ACS Synthetic Biology, 2013, 2, 537-41). This strain enables the efficient transformation and screening of pore-forming membrane peptides, nanopores and ions channels that are introduced via pACYC2, pTeT7Woder pCTRL2.

### Overview of Fluorescent Protein Sensors, Nanopores and Ion Channels

### Overview of Fluorescent Protein Sensors:

R-GECO-1: Red fluorescent Ca²⁺-specific protein sensor (Science, 2011, 333, 1888-1891). Is preferably used in the microtiter plate and in the colony format as genetically encoded reporter to detect the inflow of Ca²⁺ after the formation of a pore in the inner membrane of E. coli.

G-GECO-1: Green fluorescent Ca²⁺-specific protein sensor (Science, 2011, 333, 1888-1891). Is preferably used in the flow cytometer as genetically encoded reporter to detect the inflow of Ca²⁺ after the formation of a pore in the inner membrane of E. coli.

iGluSnFR: Denotes a class of fluorescent protein sensors specifically for L-Glu (Nature Methods, 2013, 10, 162-70). Used to detect low-molecular materials and substances beyond Ca²⁺ or to cause nanopore-dependent change in the fluorescent signal.

Overview of Membrane Proteins. Ion Channels, and Pore-Forming Membrane Peptides:

### Holins and Pinholins:

S²¹-68: Wild-type of the S²¹-68 pinholin of bacteriophage P21 with pronounced and defined pore-forming properties (PNAS, 2009, 106, 18966-18971). Exact mechanism of pore formation is currently unclear. Presumed is first an inactive conformation in the form of an antiparallel a-helix with respectively cytosolic N- and C-termini, which collect in the inner membrane of the inner membrane of E. coli. A concentration-related increase of S²¹-68 subsequently results in flipping of the first transmembrane domain by the lipid bilayer and in pore formation. Presumed is a defined pore consisting of seven subunits and a membrane channel consisting of the second transmembrane domain, which is stabilized by homomeric interactions of the first transmembrane domain in the periplasma. (PNAS, 2013, 110, E2054-E2063)

Polypeptide Sequence:

(SEQ ID NO: 1)
MDKISTGIAYGTSAGSAGYWFLQWLDQVSPSQWAAIGVLGSLVLGFL
TYLTNLYFKIREDRRKAARGE

S²¹-68 truncations: N-terminal truncations of the wild-type of the S²¹-68 pinholin of bacteriophage P21.

The polypeptide sequences are summarized in the following table:

| Trunc- | |
|---|---|
| ation | Amino acid sequence |
| S2166 | MKISTGIAYGTSAGSAGYWFLQWLDQVSPSQWAAIGVLGSL |
| | VLGFLTYLTNLYFKIREDRRKAARGE (SEQ ID NO: 28) |
| S2164 | MSTGIAYGTSAGSAGYWFLQWLDQVSPSQWAAIGVLGSLV |
| | LGFLTYLTNLYFKIREDRRKAARGE (SEQ ID NO: 29) |
| S2162 | MGIAYGTSAGSAGYWFLQWLDQVSPSQWAAIGVLGSLVLG |
| | FLTYLTNLYFKIREDRRKAARGE (SEQ ID NQ: 30) |
| S2160 | MAYGTSAGSAGYWFLQWLDQVSPSQWAAIGVLGSLVLGFL |
| | TYLTNLYFKIREDRRKAARGE (SEQ ID NO: 31) |
| S2158 | MGTSAGSAGYWFLQWLDQVSPSQWAAIGVLGSLVLGFLTY |
| | LTNLYFKIREDRRKAARGE (SEQ ID NO: 32) |
| S2156 | MSAGSAGYWFLQWLDQVSPSQWAAIGVLGSLVLGFLTYLT |
| | NLYFKIREDRRKAARGE (SEQ ID NO: 33) |
| S2154 | MGSAGYWFLQWLDQVSPSQWAAIGVLGSLVLGFLTYLTNLY |
| | FKIREDRRKAARGE (SEQ ID NO: 34) |
| S2152 | MAGYWFLQWLDQVSPSQWAAIGVLGSLVLGFLTYLTNLYFK |
| | IREDRRKAARGE (SEQ ID NO: 35) |
| S2150 | MYWFLQWLDQVSPSQWAAIGVLGSLVLGFLTYLTNLYFKIR |
| | EDRRKAARGE (SEQ ID NO: 36) |
| S2148 | MFLQWLDQVSPSQWAAIGVLGSLVLGFLTYLTNLYFKIRED |
| | RRKAARGE (SEQ ID NO: 37) |
| S2146 | MQWLDQVSPSQWAAIGVLGSLVLGFLTYLTNLYFKIREDRR |
| | KAARGE (SEQ ID NO: 38) |
| S2144 | MLDQVSPSQWAAIGVLGSLVLGFLTYLTNLYFKIREDRRKA |
| | ARGE (SEQ ID NO: 39) |
| S2142 | MQVSPSQWAAIGVLGSLVLGFLTYLTNLYFKIREDRRKAAR |
| | GE (SEQ ID NQ: 40) |
| S2140 | MSPSQWAAIGVLGSLVLGFLTYLTNLYFKIREDRRKAARGE |
| | (SEQ ID NO: 41) |
| S2138 | MSQWAAIGVLGSLVLGFLTYLTNLYFKIREDRRKAARGE |
| | (SEQ ID NO: 42) |

S²¹-71: Wild-type of the S²¹-71 anti-pinholin of bacteriophage P21. In comparison to S²¹-68, the N-terminus is expanded by three additional amino acids MKS, which limit the pore-forming properties. The exact mechanism as to how the pore formation is limited is still unclear according to current knowledge (Current Opinion in Microbiology, 2013, 16, 790-797). Presumed is a role of positive charges that prevent flipping of the first transmembrane domain.

Polypeptide Sequence:

(SEQ ID NO: 2)
MKSMDKISTGIAYGTSAGSAGYWFLQWLDQVSPSQWAAIGVLGSLVLGF
LTYLTNLYFKIREDRRKAARGE

S²¹-71 M4A: Mutant of the S²¹-71 anti-pinholin in which the second methionine is mutated to alanine at the fourth position. Thus, in principle, prevents the expression of the wild-type S²¹-68 pinholin, which in the case of S²¹-71 can also be expressed in principle by moving the translation initiation site by three amino acids. In comparison to the S²¹-71 anti-pinholin, the pore formation is thus further delayed.

Polypeptide Sequence:

(SEQ ID NO: 3)
MKSADKISTGIAYGTSAGSAGYWFLQWLDQVSPSQWAAIGVLGSLVLGF
LTYLTNLYFKIREDRRKAARGE

N-terminal S²¹-71 mutants: N-terminal mutants of the S²¹-71 anti-pinholin with the following polypeptide sequence:

MXXXDKISTGIAYGTSAGSAGYWFLQWLDQVSPSQWAAIGVLGSLVLGFLT YLTNLYFKIREDRRKAARGE (SEQ ID NO:43), wherein A, R, N, D, Q, E, G, H, I, L, K, F, S, T, W, Y or V can be provided independently of one another at each of the positions indicated by “X.”

Particularly Preferred Sequences are:

Clone P14E5:
(SEQ ID NO: 44)
MYLEDKISTGIAYGTSAGSAGYWFLQWLDQVSPSQWAAIGVLGSLVLGF
LTYLTNLYFKIREDRRKAARGE
Clone P14C11:
(SEQ ID NO: 45)
MNFEDKISTGIAYGTSAGSAGYWFLQWLDQVSPSQWAAIGVLGSLVLGF
LTYLTNLYFKIREDRRKAARGE
Clone P15D3:
(SEQ ID NO: 46)
MDWDDKISTGIAYGTSAGSAGYWFLQWLDQVSPSQWAAIGVLGSLVLGF
LTYLTNLYFKIREDRRKAARGE
Clone P7D10:
(SEQ ID NO: 47)
MAGWDKISTGIAYGTSAGSAGYWFLQWLDQVSPSQWAAIGVLGSLVLGF
LTYLTNLYFKIREDRRKAARGE
Clone P3G3:
(SEQ ID NO: 48)
MWRGDKISTGIAYGTSAGSAGYWFLQWLDQVSPSQWAAIGVLGSLVLGF
LTYLTNLYFKIREDRRKAARGE
Clone P3E5:
(SEQ ID NO: 49)
MFQWDKISTGIAYGTSAGSAGYWFLQWLDQVSPSQWAAIGVLGSLVLGF
LTYLTNLYFKIREDRRKAARGE

^{SGS}ATMD1-S²¹68: Fragment of the S²¹-68 pinholin in which the first transmembrane domain ATMD1 was deleted. Expression is in principle toxic, presumably due to the ability to form at least transient pores in the inner membrane of E. coli (PNAS, 2009, 106, 18966-18971; Molecular Microbiology, 2010, 76, 68-77).

Polypeptide Sequence:

(SEQ ID NO: 4)
MSGSMWAAIGVLGSLVLGFLTYLTNLYFKIREDRRKAARGE

^{TVMV}ΔTMD1-S²¹68: Fragment of the S²¹-68 pinholin in which the first transmembrane domain TMD1 is missing and the cutting sequence ETVRFQ'S precedes the TVMV protease N-terminal. Expression is non-toxic, presumably likely due to the proteolytic TVMV interface, which greatly limits the pore-forming properties.

Polypeptide Sequence:

(SEQ ID NO: 5)
MSSSGGSETVRFQSGSMWAAIGVLGSLVLGFLTYLTNLYFKIREDRRKA
ARGE

S¹⁰⁵: Wild-type of the S¹⁰⁵ pinholin of bacteriophage A with pronounced pore-forming properties (PNAS, 2010, 107, 2219-23). In contrast to the S²¹-68 pinholin, it is presumed that the S105 holin forms large, pm-large pores. However, the exact mechanism is unclear according to current knowledge.

Polypeptide Sequence:

(SEQ ID NO: 6)
MPEKHDLLAAILAAKEQGIGAILAFAMAYLRGRYNGGAFTKTVIDATMC
AIIAWFIRDLLDFAGLSSNLAYITSVFIGYIGTDSIGSLIKRFAAKKAG
VEDGRNQ

S¹⁰⁷: Wild-type of the S¹⁰⁷ anti-pinholin of bacteriophage A. Analogously to the S²¹-71 anti-pinholin, the pore-forming properties are greatly limited due to two additional amino acids MK at the N-terminus. However, an exact mechanism is unclear according to current knowledge (Molecular Microbiology, 1993, 8, 525-33).

Polypeptide Sequence:

(SEQ ID NO: 7)
MKMPEKHDLLAAILAAKEQGIGAILAFAMAYLRGRYNGGAFTKTVIDAT
MCAIIAWFIRDLLDFAGLSSNLAYITSVFIGYIGTDSIGSLIKRFAAKK
AGVEDGRNQ

S¹⁰⁷-M3A: Mutant of the S¹⁰⁷ anti-pinholin in which the second methionine is mutated to alanine at the third position. Thus, in principle, prevents the expression of the wild-type S¹⁰⁵ holin, which in the case of S¹⁰⁷ can also be expressed in principle by moving the translation initiation site by 3 amino acids. In comparison to the S¹⁰⁷ anti-pinholin, the pore formation is thus further delayed.

Polypeptide Sequence:

(SEQ ID NO: 8)
MKAPEKHDLLAAILAAKEQGIGAILAFAMAYLRGRYNGGAFTKTVIDAT
MCAIIAWFIRDLLDFAGLSSNLAYITSVFIGYIGTDSIGSLIKRFAAKK
AGVEDGRNQ

T4 pinholin: Wild-type of the T4 pinholin of bacteriophage T4 with highly pronounced pore-forming properties. The T4 pinholin has a transmembrane domain with a long periplasmic C-terminus and a short cytosolic N-terminus. According to the literature, both termini are regulated by an anti-holin mechanism, which runs completely differently than in the previously known holins S¹⁰⁵ and S²¹68 (Journal of Bacteriology, 2016, 198, 2448-57). That is to say, no flipping of individual transmembrane helices is currently being postulated. However, currently, the exact mechanism remains unclear.

Polypeptide Sequence:

(SEQ ID NO: 9)
MAAPRISFSPSDILFGVLDRLFKDNATGKVLASRVAVVILLFIMAIVWY
RGDSFFEYYKQSKYETYSEIIEKERTARFESVALEQLQIVHISSEADFS
AVYSFRPKNLNYFVDIIAYEGKLPSTISEKSLGGYPVDKTMDEYTVHLN
GRHYYSNLKFAFLPTKKPTPEINYMYSCPYFNLDNIYAGTITMYWYRND
HISNDRLESICAQAARILGRAK

T4^{ΔC-Tail}: Truncated version of the T4 pinholin in which the periplasmically located C-terminus was deleted. This variant is non-toxic. Mechanism unknown so far.

Polypeptide Sequence:

(SEQ ID NO: 10)
AAPRISFSPSDILFGVLDRLFKDNATGKVLASRVAVVILLFIMAIVWY

^{ΔN-Tail}T4^{ΔC-Tail}: Truncated version of the T4 pinholin, which only has the transmembrane domain. This variant is toxic, mechanism unknown.

Polypeptide Sequence:

(SEQ ID NO: 11)
MVAVVILLFIMAIVWY

### Ion Channels

K_{CV}^{NTS}: Viral potassium channel that specifically conducts potassium, but also allows calcium to pass through. (BBA-Biomembranes, 2014, 1838, 1096-1103).

Polypeptide Sequence:

(SEQ ID NO: 12)
MLLLIIHLSILVIFTAIYKMLPGGMFSNTDPTWVDCLYFSASTHTTVGY
GDLTPKSPVAKLTATAHMLIVFAIVISSFTFPW

K_{CV}^{NTS G77S}: Based on K_{CV}^{NTS} with a mutation of glycine to serine at position 77. This mutation results in an average open probability and thus to a reduction in the conductivity of the potassium channel.

Polypeptide Sequence:

(SEQ ID NO: 13)
MLLLIIHLSILVIFTAIYKMLPGGMFSNTDPTWVDCLYFSASTHTTVGY
GDLTPKSPVAKLTATAHMLIVFAIVISGFTFPW

K_{CV}^{PBCV1}: Viral potassium channel that specifically conducts potassium, but also allows calcium to pass through. Very low open probability and thus low conductivity (FEBS Lett, 2003, 552, 12-6).

Polypeptide Sequence:

(SEQ ID NO: 14)
MLVFSKFLTRTEPFMIHLFILAMFVMIYKFFPGGFENNFSVANPDKKAS
WIDCIYFGVTTHSTVGFGDILPKTTGAKLCTIAHIVTVFFIVLTL

### Others

HokB: Pore-forming membrane peptide; forms part of a bacterial efflux or toxin/antitoxin system. Specifically forms HokB nanopores in the inner membrane and thus enables the unspecific outflow of toxic substances, such as antibiotics (MBio. 2018, 9, pii: e00744-18).

Polypeptide Sequence:

(SEQ ID NO: 15)
MKHNPLVVCLLIICITILTFTLLTRQTLYELRFRDGDKEVAALMACTSR

TisB: Pore-forming membrane peptide; forms part of a bacterial efflux or toxin/antitoxin system. Specifically forms TisB nanopores in the inner membrane and thus enables the unspecific outflow of toxic substances, such as antibiotics (Biophysical Journal, 2012, 103, 1460-1469).

Polypeptide Sequence:

(SEQ ID NO: 16)
MNLVDIAILILKLIVAALQLLDAVLKYLK

αHLA: a-hemolysine refers to a pore-forming toxin from S. aureus (PNAS, 2008, 105, 19720-19725). Used as prototypic nanopore for a number of studies and projects in the area of nanopore engineering, especially in the development of sensory applications. Such projects are however almost exclusively realized by the expression of αHLA in cell-free systems. Functional states are investigated either by means of high-resolution biophysical methods or with blood cells as a substrate. Is in principle exported extracellularly, even in E. coli.

Polypeptide Sequence:

(SEQ ID NO: 17)
MKIRIVSSVIIILLLGSILMNPVAGAADSDINIKIGIIDIGSNIIVKIG
DLVIYDKENGMHKKVFYSFIDDKNHNKKLLVIRTKGTIAGQYRVYSEEG
ANKSGLAWPSAFKVQLQLPDNEVAQISDYYPRNSIDTKEYMSTLTYGFN
GNVTGDDTGKIGGLIGANVSIGHTLKYVQPDFKTILESPTDKKVGWKVI
FNNMVNQNWGPYDRDSWNPVYGNQLFMKTRNGSMKAADNFLDPNKASSL
LSSGFSPDFATVITMDRKASKQQTNIDVIYERVRDDYQLHWTSTNWKGT
NTKDKWTDRSSERYKIDWEKEEMTN

cWZA: Minimum pore-forming membrane peptide artificially derived from the transmembrane region of the membrane protein Wza (Nature Chemistry, 2017, 9, 411-419). In reconstituted lipid membrane bilayers, cWZA has been shown to form defined heptameric pores; but relies on ring-shaped low-molecular substances that associate with the individual membrane peptides in order to form stable or defined nanopores in the membrane.

Polypeptide Sequence:

(SEQ ID NO: 18)
MAPLVRWNRVISQLVPTITGVHDLTETVRYIKTWPN

BM2: Proton channel from the influenza B virus (Chemical Sciences, 2018, 9, 2365-2375).). Permeable exclusively to protons, depolarizes the inner membrane of E. coli, results in cell death. Used as control for checking the specificity of pore-related inflow of Ca²⁺ in various screening formats.

Polypeptide Sequence:

(SEQ ID NO: 19)
MLEPFQILSISSFILSALHFIAWTIGHLNQIKR

HCV TME1 and TME2: Transmembrane domains of the hepatitis C virus shell proteins, which bind to the membrane and act toxically. (Biochim Biophys Acta, 2004, 28; 1660(1-2):53-65)

Polypeptide Sequences:

HCV TME1:
(SEQ ID NQ: 20)
MIAGAHWGVLAGIAYFSMVGNWAKVLVVLLLFAGVDA
HCV TME2:
(SEQ ID NO: 21)
MEYVVLLFLLLADARVCSCLWMMLLISQAEA

### Example 1: Validation of the Fluorescence Assay in Microtiter Plates

### Background:

First, the newly developed method was validated in E. coli in microtiter plate format. In particular, there should be a clarification of the extent to which the inflow of Ca²⁺ as a function of the expression of a pore in the inner membrane of E. coli is associated with an increase in fluorescence. The model peptides were the pore-forming membrane peptide ^{SGS}ΔTMD1-S²¹68 and the non-pore-forming membrane peptide ^{TVMV}ΔTMD1-S²¹68 as positive or negative control.

### Experimental Procedure and Results:

1. Co-Transformation of the R-GECO1 and the Two Pinholin Fragments in Chemically Competent E. coli.

Transformed individually in each case:

→BL21(DE3) with pPRO24-R-GECO1 (reporter)+pACYCT2-^{SGS}ΔTMD1-S²¹68 (positive)
→BL21(DE3) with pPRO24-R-GECO1 (reporter)+pACYCT2-^{TVMV}ΔTMD1-S²¹68 (negative)
→BL21(DE3) with pPRO24-R-GECO1 (reporter)

### 2. Inoculation Overnight:

Colonies of BL21(DE3) on LB agar from 1. were picked and cultivated in deep well plates with 300 μl of LB medium with the necessary antibiotics overnight for about 16 h at 37° C. and 1300 rpm.

### 3. Microtiter Plate Readout:

After cultivating individual clones from 2. overnight, the OD₆₀₀ of the precultures in microtiter plates (MTP) was adjusted to 0.1, and the samples, 200 μL each, were shaken at 37° C. and 180 rpm for 30 min.

Thereafter, the R-GECO1 was initially induced with 25 mM sodium propionate, pH 8.0, and the fluorescence was measured over 120 min.

Subsequently, the two pinholin fragments were then induced with 0.5 mM IPTG, and the fluorescence was measured over another 120 min.

General information: The measurement in the reader is carried out at 30° C. every 10 min. The plate is shaken at 180 rpm between the measurements. The OD_{600 nm} and the fluorescence (Ex: 555 nm; Em: 600 nm) are measured. The volume in the cavities is 200 μl after addition of all inductors.

### 4. Experimental Analysis:

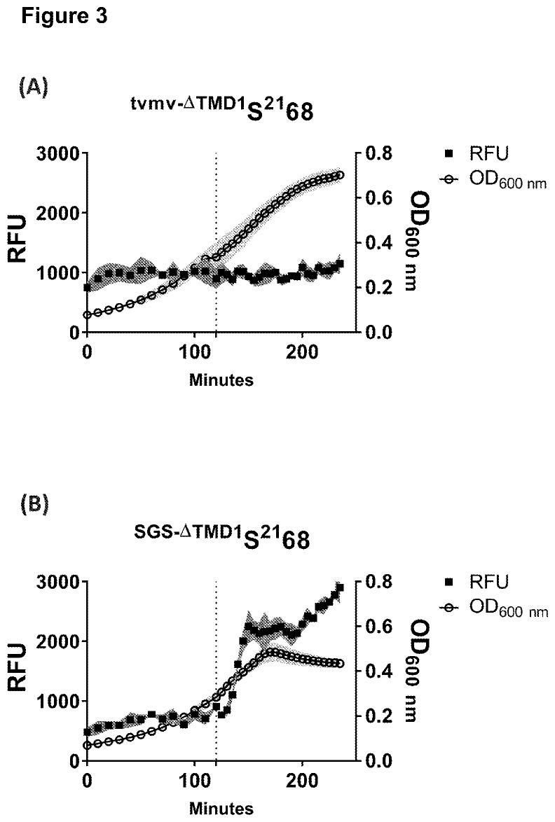

For the analysis, the data of three different colonies were averaged and the OD_{600 nm} and in relative fluorescence units (RFU) were in each case plotted in a graph (FIG. 3). From the respective curves, various functions and properties can subsequently be derived: e.g., whether the expression of a nanopore has toxic effects or even results in a loss of cellular integrity and how the latter correlates in each case with the Ca²⁺-related increase in a fluorescent signal. Here, further parameters, such as the half-maximum time of the fluorescence signal or its slope, can be used and quantified.

### Conclusion:

Overall, pore-forming membrane peptides can be quantitatively imaged in microtiter plate format by means of the newly developed genetic assay in E. coli and can be differentiated from the non-pore-forming membrane peptides. Various parameters can be used to quantitatively characterize the pore-forming properties of a membrane peptide, a nanopore or an ion channel (FIG. 3). This includes, among other things, the time-dependent inflow of Ca²⁺ (and associated slope of the fluorescence increase or the half-maximum time of the fluorescence signal) along with the OD600: The intensity of the fluorescence signal inter alia reflects the ability of a membrane peptide to form pores in the inner membrane; and the OD600 gives complementary information about the toxicity of a pore-forming peptide and about the cellular integrity.

### Example 2: Investigation of the Pore-Forming Properties of Various Membrane Peptides, Nanopores and Ion Channels in Microtiter Plates

### Background:

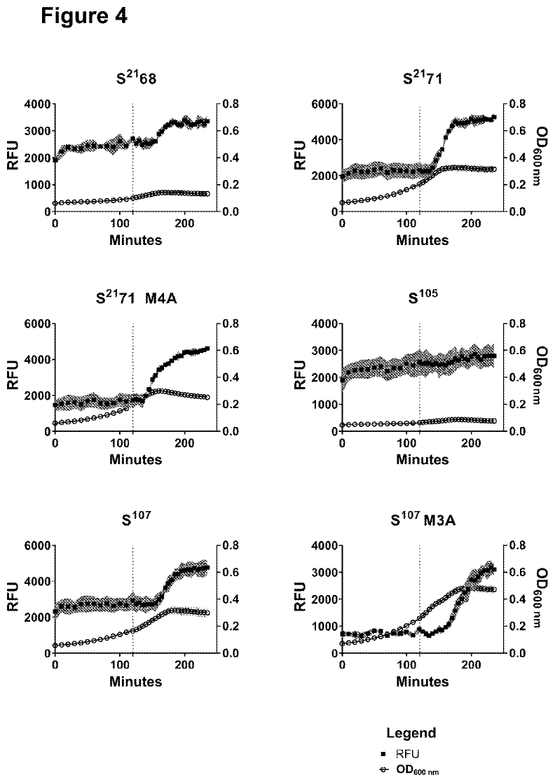

It was then clarified whether or how easily the developed method can be applied to further pore-forming membrane peptides, nanopores and ion channels. For this purpose, we tested 19 different variants derived from a total of 10 different classes of nanopores or ion channels with respect to an expression-related increase in the fluorescent signal in E. coli. Measurements were basically carried out in the microtiter plate format. The results are summarized (FIG. 4) with brief explanations in each case (Table 1).

### Experimental Procedure and Results:

1. Co-Transformation of the R-GECO1 and the Pore-Forming Proteins in Chemically Competent E. coli.

Transformed individually in each case:

→BL21(DE3) with pPRO24-R-GECO1 (reporter)+pTeT7W-“nanopore” (Table 1).

### 2. Inoculation Overnight:

Colonies of BL21(DE3) on LB agar from 1. were picked and cultivated in deep well plates with 300 μl of LB medium with the necessary antibiotics overnight for about 16 h at 37° C. and 1300 rpm.

### 3. Microtiter Plate Readout:

After cultivating individual clones from 2. overnight, the OD₆₀₀ of the precultures in microtiter plates (MTP) was adjusted to 0.1, and the samples, 200 μL each, were shaken at 37° C. and 180 rpm for 30 min.

Thereafter, the R-GECO1 was initially induced with 25 mM sodium 25 propionate, pH 8.0, and the fluorescence was measured over 120 min.

Subsequently, the pores were then induced with 0.5 mM IPTG, and the fluorescence was measured over another 120 min.

General information: The measurement in the reader is carried out at 30° C. every 10 min. The plate is shaken at 180 rpm between the measurements. The OD_{600 nm} and the fluorescence (Ex: 555 nm; Em: 600 nm) are measured. The volume in the cavities is 200 μl after addition of all inductors.

### 4. Experimental Analysis:

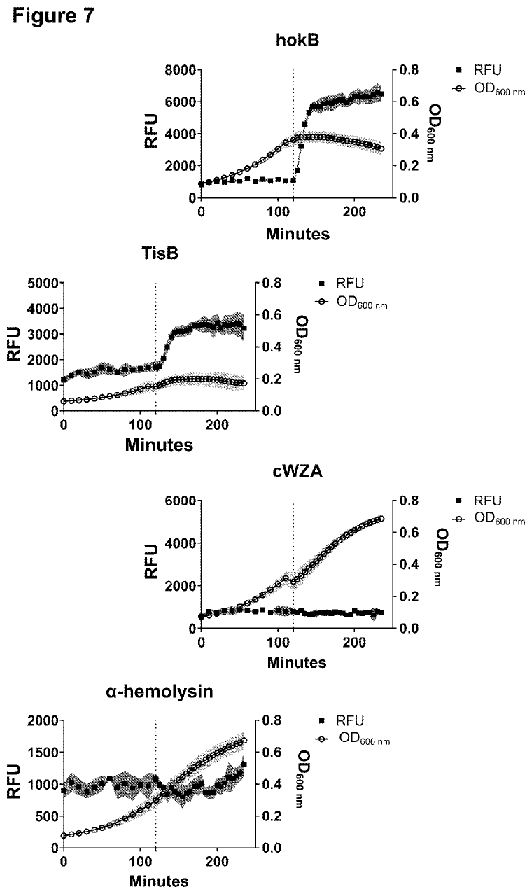

For the analysis, the data of three different colonies were averaged and the OD_{600 nm} and in the fluorescence in relative fluorescence units (RFU) were in each case plotted in a graph (FIGS. 4-8). Various properties can then be derived from the respective curves. Depending on the course of the curve, different functions and properties can be derived: e.g., whether the expression of a nanopore has toxic effects or even results in a loss of cellular integrity and how the latter correlates in each case with the Ca²⁺-related increase in a fluorescent signal.

### Conclusion:

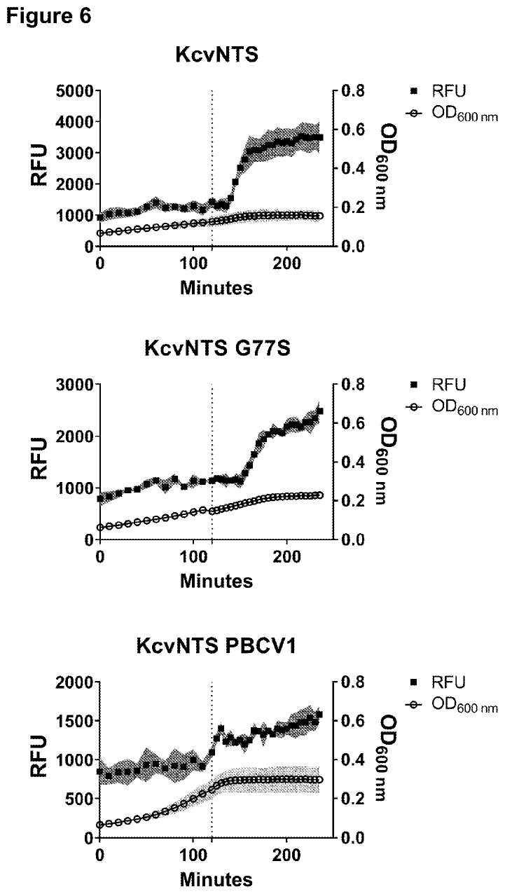

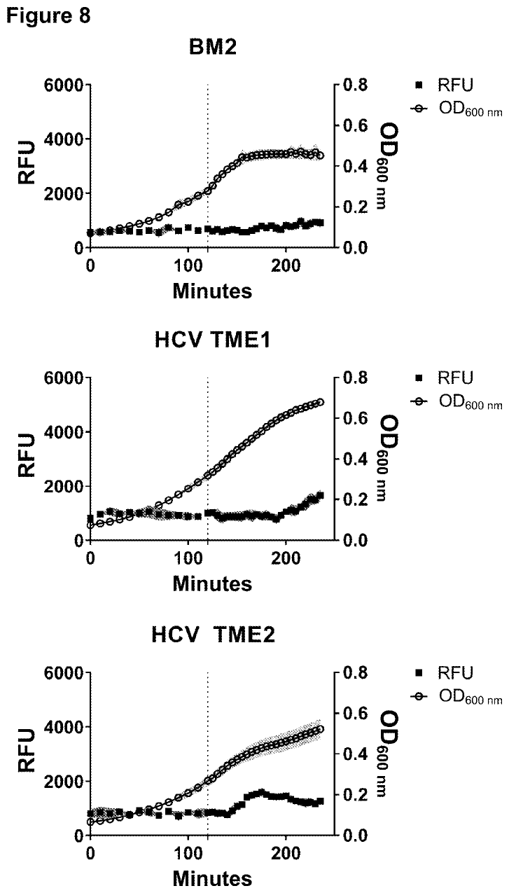

In summary, a total of 20 variants of 10 different nanopores, ion channels and membrane peptides were successfully measured with the method (FIG. 3. and FIGS. 4-8). The experiments show that the inflow of Ca²⁺ and thus an increase in the fluorescent signal takes place exclusively as a function of the pore formation and not, for example, by pore-related toxic effects which impair the integrity of the cell membrane. Among others, this also includes the proton channel BM2 derived from the influenza B virus, which is demonstrably permeable only to protons, but not Ca²⁺, as a negative control and transmembrane peptides from the hepatitis C virus, the expression of which is toxic to E. coli, but does not result in an inflow of Ca²⁺ (FIG. 8). In addition, it was demonstrated that K⁺-specific K_{CV} channels have a promiscuous permeability with respect to Ca²⁺ ions, which result in differently high fluorescent signals depending on the opening state (FIG. 6). In particular, the method can thus find broad application for quantitatively measuring the pore-forming properties or flow specificity of ion channels and nanopores in E. coli.

TABLE 1
Overview of the various classes of nanopores and ion channels
that were investigated by means of the novel method.

| Nanopore | Nanopore | |
|---|---|---|
| System | Variant | Results/Comments |
| S²¹ | S²¹-68 | Wild-type S21-68 pinholin. Shows |
| | | pronounced pore formation. |
| | S²¹-71 | Shows limited pore formation in |
| | | comparison to S²¹-68. |
| | S²¹-71 M4A | M4A mutation prevents expression |
| | | of the wild-type S²¹-68 |
| | | pinholin moving the translation |
| | | initiation screen and thus shows |
| | | greatly limited pore formation in |
| | | comparison to S²¹-68 and S²¹-71. |
| | SGS- | The second transmembrane |
| | ^{ΔTMD1}S²¹ | domain of the S²¹-68 pinholin |
| | | shows pronounced pore formation. |
| | TVMV- | A preceding TVMV interface |
| | ^{ΔTMD1}S²¹ | severely limits pore formation. |
| S¹⁰⁵ | S¹⁰⁵ | Wild-type holin of bacteriophage A |
| | | with pronounced pore formation. |
| | S¹⁰⁷ | Wild-type anti-holin of bacteriophage A |
| | | with limited pore-forming properties. |
| | S¹⁰⁷-M3A | M3A mutation prevents expression of |
| | | the wild-type S105 pinholin |
| | | moving the translation initiation |
| | | screen and thus shows greatly |
| | | limited pore formation in comparison |
| | | to S105 and S107. |
| T4 holin | Wild-type | Wild-type holin of the bacteriophage |
| | | T4 with pronounced pore formation. |
| | T4^{ΔC-Tail} | Truncated version of the T4 holin |
| | | in which the periplasmically positioned |
| | | C-terminus was deleted, with limited |
| | | pore formation. |
| | ^{ΔN-Tail}T4^{ΔC-Tail} | Truncated version of the T4 holin, |
| | | which only has the transmembrane |
| | | domain, with strongly pronounced |
| | | pore formation. |
| K_{CV} | PBCV1 | Variant of a K⁺-specific ion channel |
| | | with a promiscuous permeability |
| | | to Ca²⁺ depending on the opening |
| | | probability of the ion channel |
| | NTS G77S | Variant of a K⁺-specific ion channel |
| | | with a promiscuous permeability |
| | | to Ca²⁺ depending on the opening |
| | | probability of the ion channel |
| | NTS | Variant of a K⁺-specific ion channel |
| | | with a promiscuous permeability |
| | | to Ca²⁺ depending on the opening |
| | | probability of the ion channel |
| Nanopores | HokB | Pore-forming membrane peptide shows |
| from toxin/ | | pronounced pore-forming properties. |
| antitoxin | TisB | Pore-forming membrane peptide shows |
| systems | | pronounced pore-forming properties. |
| αHLA | | Pore-forming toxin, but does not result |
| | | in pore formation within the scope of |
| | | the method. Presumably because is it |
| | | directly exported from E. coli |
| | | via defined export signals: i.e., there |
| | | is no canonical export sequence |
| | | (e.g., no PelB leader sequence). |
| cWZA | | Artificial pore-forming membrane peptide, |
| | | which forms artificial nanopores only |
| | | in the context of in-vitro lipid bilayer |
| | | experiments, but does not result in |
| | | functional pore formation in the context of |
| | | the genetic screening method. |
| BM2 | | NEGATIVE CONTROL: Pore-forming |
| | | membrane peptides derived from |
| | | the influenza B virus and only |
| | | permeable to protons. Demonstrably |
| | | results in cell death on account of |
| | | the pore formation, but as expected |
| | | not in an increase in the Ca2+-dependent |
| | | fluorescence. |
| HCV | TME1 | Transmembrane domains of the hepatitis |
| TMEs | | C virus shell proteins, which bind to |
| | | the membrane and act toxic |
| | TME2 | Transmembrane domains of the hepatitis |
| | | C virus shell proteins, which bind to |
| | | the membrane and act toxic |

### Example 3: Model Selection Experiments in Microtiter Plates

### Background:

An important aspect in the development of screening methods is to perform a check of the extent to which a functional variant of a larger amount of non-functional variants can be identified, selected and enriched. This is usually detected in model selections in which the DNA of a functional protein is diluted relative to a non-functional protein and then reidentified by means of a functional assay.

In the course of a model selection, it is thus comprehensively demonstrated that the connection between genotype and phenotype, i.e., the physical association or assignment between the function of a protein and its coding DNA is maintained during the entire screening cycle. In the context of pore-forming membrane peptides, there must also be a check as to what extent toxic effects associated with the pore-forming properties of the membrane peptides, for example during growth due to non-specific expression, i.e., before they are subjected to a functional test, are negatively enriched or a selection against the desired properties takes place.

The model peptides were, as already in Example 1, ^{SGS}ATMD1-S²¹68 as pore-forming and ^{TVMV}ATMD1-S²168 as non-pore-forming membrane peptide.

### Experimental Procedure and Results:

1. Co-Transformation of the R-GECO1 and the Two Pinholin Fragments in Chemically Competent E. coli. The R-GECO1 could be Expressed as Previously in pPRO24 and the Pinholin Fragments in pTeT7W.

The following combinations were tested as dilution in each case:

→In a ratio of 1:5 of ^{SGS}ΔTMD1-S²¹68 to ^{TVMV}ΔTMD1-S²¹68+R-GECO1
→In a ratio 1:10 of ^{SGS}ATMD1-S²¹68 to ^{TVMV}ΔTMD1-S²¹68+R-GECO1

### 2. Inoculation Overnight:

Colonies with ratios of 1:5 and 1:10 were randomly picked and cultivated in 25 deep well plates with 500 μl of LB medium with the necessary antibiotics overnight for about 16 h at 30° C. and 1300 rpm.

For this purpose, 89 colonies were picked in each case

In addition, per 96-well microtiter plate, 3 positive controls and 3 negative controls were picked in a targeted manner

1 cavity was filled only with LB medium as control

The deep well plate was stored at 4° C.

### 3. Preparation for Microtiter Plate Readout:

After cultivation from 2. overnight, the OD₆₀₀ of the precultures in microtiter plates (MTP) was adjusted to about 0.1, and the samples, 200 μL each, were shaken at 37° C. and 180 rpm for 30 min. It was assumed that all others have a similar OD_{600 nm} and that all samples can thus be diluted identically.

Thereafter, the R-GECO1 was initially induced with 25 mM sodium propionate, pH 8.0, and the fluorescence was measured over 120 min.

General information: The measurement in the reader is carried out at 30° C. every 10 min. The plate is shaken at 180 rpm between the measurements. The OD_{600 nm} and the fluorescence (Ex: 555 nm; Em: 600 nm) are measured. The volume in the cavities is 200 μl.

### 4. Experimental Analysis

For the analysis, the data of each cavity, i.e., the OD_{600 nm} and the fluorescence in relative fluorescence units (RFU) were plotted separately in each case. For normalization and better clarity, the fluorescence can also be divided by the OD₆₀₀ independently of the toxicity. For a more clearly arranged analysis, end point measurements are shown below:

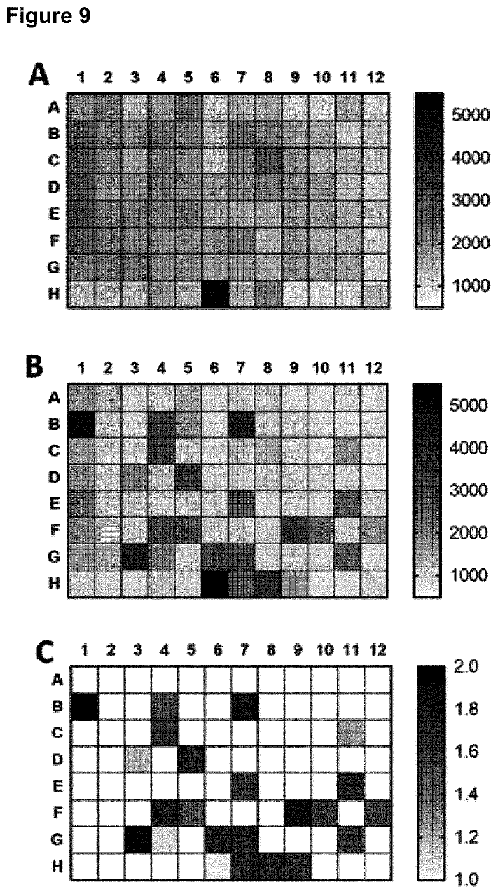

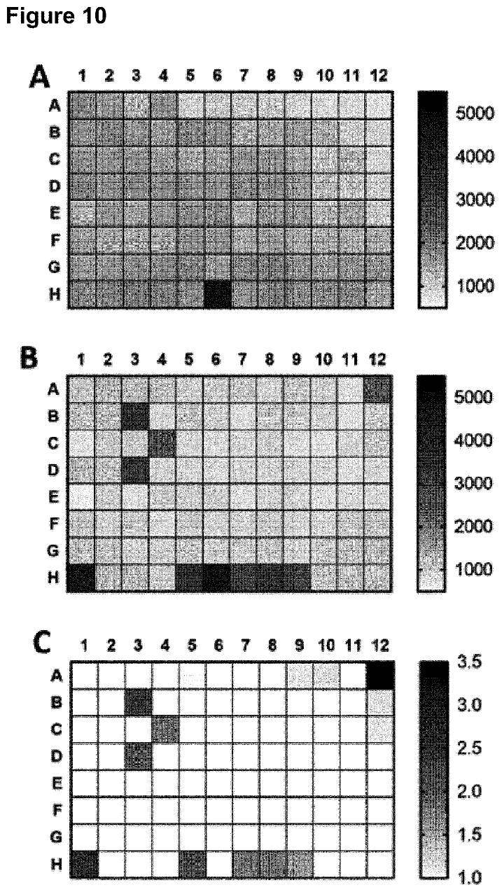

→120 min after sodium propionate, but before IPTG addition and induction of the pore (FIG. 9A and FIG. 10A).
→120 min after IPTG addition and induction of the pore (FIG. 9B and FIG. 10B)

Subsequently, the clones with the highest fluorescence values were reinoculated in the deep well plate, which was stored at 4° C., and the DNA was isolated. In all colonies, sequencing resulted in the positive control pACYCT2-^{SGS}ΔTMD1-S²¹68.

→In the 1:5 dilution (FIG. 9C) from B1, B4, B7, C4, D5 and E7.
→In the 1:10 dilution (FIG. 10C) from A12, B3, C4, D3, H1 and H5.

Optionally, it is possible to change the threshold value in both model selections and thus to select for different properties. In addition, the kinetics (i.e., the half-maximum time of the fluorescence signal or the slope) can also be analyzed during the pore formation, which is visible immediately after induction of the nanopore.

### Conclusion:

Overall, by means of the newly developed method, the DNA of the pore-forming membrane peptide ^{SGS}ΔTMD1-S²¹68 can be reidentified from a five- or ten-fold dilution to the non-pore-forming peptide ^{TVMV}ΔTMD1-S²¹68 in correspondingly equal proportions within a selection cycle in the microtiter plate format, selected and thus enriched. On the one hand, this demonstrates that the connection between genotype and phenotype is maintained within the selection cycle and that under the given circumstances, no counter-selection of the desired properties takes place, for example due to the toxicity of the pore-forming peptides.

### Example 4: Selection Experiments for Finding New Properties and Functions of Pore-Forming Membrane Peptides in Microtiter Plates

### Background:

In addition to model selections, a library of pore-forming variants of the wild-type pinholin S²¹68 in the microtiter plate format was also screened for new properties or functions. The primary aim of a real selection is to perform a check of the extent to which a screening method can be used to identify 30 novel properties or functions of pore-forming membrane peptides. In addition, i.e., analogously to the model selection experiments with the two pinholin variants ^{TVMV}ΔTMD1-S²¹68 and ^{SGS}ATMD1-S²¹68 (see Example 3), there must be a check of the extent to which the pore-forming properties in the context of a larger library have an effect on the relative frequency of variants during growth, i.e., before a functional test is carried out.

Specifically, the three N-terminal amino acids of the natural S²¹71 pinholin were randomized by means of saturation mutagenesis in order to check which mutations have a positive or negative effect on the pore-forming properties of the wild-type S²¹71 pinholin.

### Experimental Procedure and Results:

### 1. Design of the Mutation Primer

The mutagenesis primer for the S²¹71 anti-holin designed for the following base pair sequence or amino acid sequence:

Amino acids:
ATG AAA TCT ATG GAC AAA ATC TCA ACT
M K S M D K I S T
Primer:
(SEQ ID NO: 22)
TGATGAGG CAT ATG NNK NNK NNK GAC AAA ATC TCA ACT

### 2. Cloning of the Library

Initially, the S²¹71 anti-holin was amplified by the above-mentioned forward primer and a suitable reverse primer and brought into the pCTRL2 vector with the respective interfaces NdeI/KpnI. A small model library was prepared from about 4000 clones (DH10β).

3. Co-transformation of the library in chemically competent BL21(DE3)

Transformed individually in each case

→BL21(DE3) with pPRO24-R-GECO1 (reporter)+pCTRL2-^{SGS}ΔTMD1-S²¹68 (positive)
→BL21(DE3) with pPRO24-R-GECO1 (reporter)+pCTRL2-^{TVMV}ΔTMD1-S²¹68 (negative)
→BL21(DE3) with pPRO24-R-GECO1 (Reporter)+pCTRL2-S²¹68
→BL21(DE3) with pPRO24-R-GECO1 (reporter)+pCTRL2-S²¹71
→BL21(DE3) with pPRO24-R-GECO1 (reporter)+pCTRL2-S²¹71 M4A
→BL21(DE3) with pPRO24-R-GECO1 (reporter)+pCTRL2-^{3×NNK}S²¹68 (library)

Only as a result of the specially developed pCTRL2 vector could a retransformation of the wild-type holin to this extent be ensured.

### 4. Inoculation Overnight:

Colonies of BL21(DE3) on LB agar from 3. were picked and cultivated in deep well plates with 300 μl of LB medium with the necessary antibiotics overnight for about 16 h at 37° C. and 1300 rpm.

### 5. Microtiter Plate Readout:

After cultivating individual clones from 2. overnight, the OD₆₀₀ of the precultures in microtiter plates (MTP) was adjusted to 0.1, and the samples, 200 μL each, were shaken at 37° C. and 180 rpm for 30 min.

Thereafter, the R-GECO1 was initially induced with 25 mM sodium propionate, pH 8.0, and the fluorescence was measured over 120 min.

Subsequently, the library along with the positive and negative control were then induced with 0.5 mM IPTG, and the fluorescence was measured over another 120 min.

General information: The measurement in the reader is carried out at 30° C. every 10 min. The plate is shaken at 180 rpm between the measurements. The OD_{600 nm} and the fluorescence (Ex: 555 nm; Em: 600 nm) are measured. The volume in the cavities is 200 μl after addition of all inductors.

### 6. Analysis

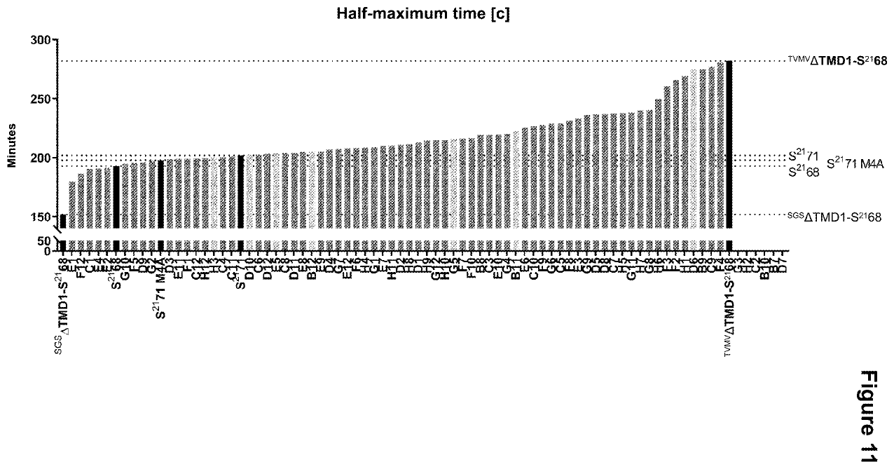

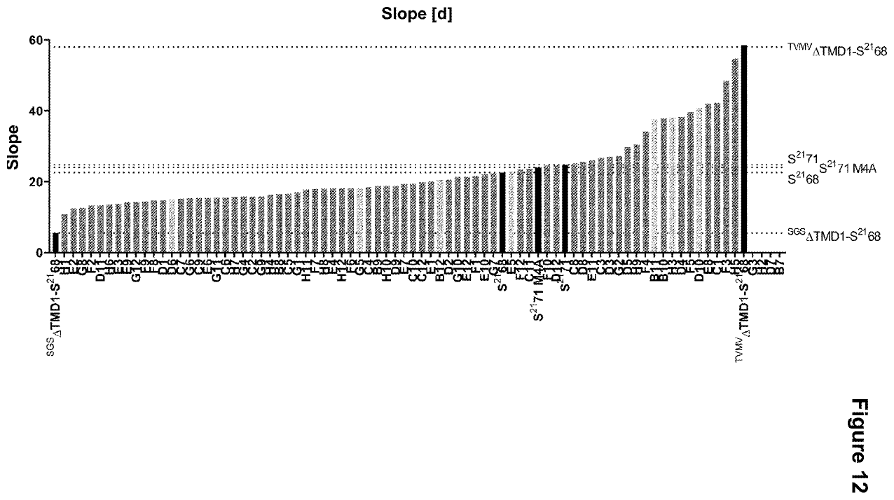

For the analysis, the curves were fitted empirically with equation 1 in order to quantitatively determine the half-maximum time c or the slope d, for example. The distribution of the parameters investigated with respect to the half-maximum time c and the slope d of the library is summarized by way of example in FIG. 11 and FIG. 12.

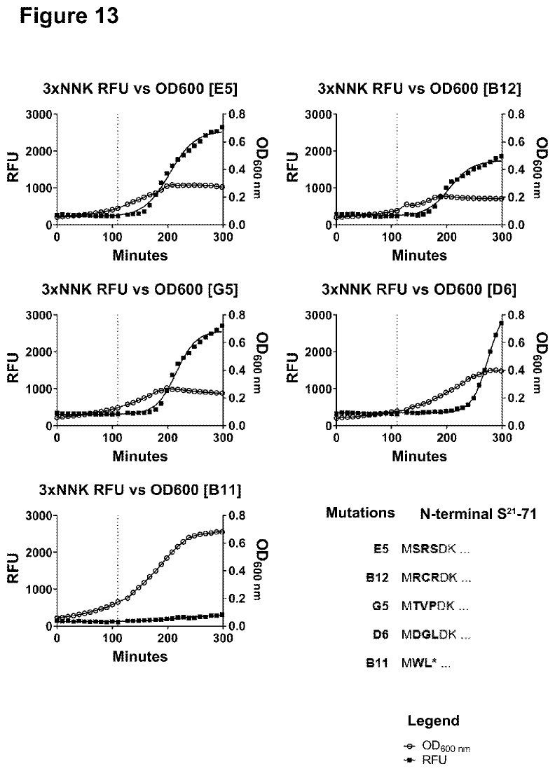

Individual mutants are subsequently sequenced and subjected to a detailed functional characterization. In this case, five individually investigated mutants show different pore-forming properties depending on the N-terminal sequences (FIG. 13).

$$f ( x ) = a + \frac{b}{1 + {e}^{\frac{- ( x - c )}{d}}} \qquad Eq . 1$$

### Conclusion:

In addition, the screening of a part of the library shows that changes in the N-terminal sequence before the first transmembrane domain (TMD) can change the pore-forming properties of the S²¹-68 pinholin. First structure/function correlations indicate that charges have a strong effect on the slope and the half-maximum time of the pore formation. The high transformation efficiency of the libraries of >10⁵ per μg DNA indicates that sufficiently large libraries of a pore-forming membrane peptide that is in principle toxic can routinely be transformed in E. coli and its pore-forming properties can be read by means of the optical reporters R-GECO1 and G-GECO1. If necessary, the expression of the pore-forming peptides in the non-induced state can be further suppressed.

### Example 5: Obtaining the Cellular Integrity or Extent of the Cell Lysis after Expression of Pore-Forming Membrane Peptides

### Background:

In principle, it is not possible to perform a check in the microtiter plate as to what extent the cell lysis takes place in each case after expression of a pore-forming membrane peptide or to what extent the cellular integrity of the 10 E. coli is preserved. Both aspects have advantages and disadvantages or develop different biotechnological applications: (a) In order to screen pore-forming membrane peptides at the single-cell level, for example by means of a flow cytometer or in the context of a microfluidic system, it is critical that the cellular integrity is preserved in order to maintain the connection between genotype and phenotype during the entire selection cycle; (b) Alternatively, the loss of cellular integrity and associated cell lysis can be used in biotechnological processes to release the cytosolic content of a microorganism, e.g., in the recombinant expression of proteins, or also in industrial biotechnology to release important metabolic products on an industrial scale.

Specifically, there was an investigation as to what extent the expression of a pore-forming peptide lyzes cells or to what extent the cellular integrity in the form of a shell is preserved. This was demonstrated by the proportion of fluorescent proteins in the soluble fraction in comparison to the non-soluble fraction. Soluble protein is conditional on digestion of the cell, wherein fluorescence in the insoluble fraction is due to cells in which cellular integrity is preserved.

### Experimental Procedure and Results:

1. Co-Transformation of R-GECO1 and the Lyzing and Non-Lyzing Pores in Chemically Competent E. coli.

Transformed individually in each case:

→BL21(DE3) with pPRO24-R-GECO1 (reporter)+pTeT7W-^{SGS}ΔTMD1-S²¹68 (non-lyzing)
→BL21(DE3) with PRO24-R-GECO1 (reporter)+pTeT7W-BM2 (non-lyzing)
→BL21(DE3) with pPRO24-R-GECO1 (reporter)+pTeT7W-S²¹71 (semi-lyzing)
→BL21(DE3) with pPRO24-R-GECO1 (reporter)+pTeT7W-S¹⁰⁷ (lyzing)
→BL21(DE3) with pPRO24-R-GECO1 (reporter)+pTeT7W-T4 (lyzing)
→BL21(DE3) with pPRO24-R-GECO1 (reporter)+pTeT7W-TisB (lyzing)
→BL21(DE3) with pPRO24-R-GECO1 (reporter)+pTeT7W-HokB (lyzing)
→BL21(DE3) with pPRO24-R-GECO1 (reporter) (negative control)

### 2. Inoculation Overnight:

Colonies of BL21(DE3) on LB agar from 1. were picked and cultivated in deep well plates with 300 μl of LB medium with the necessary antibiotics overnight for about 16 h at 37° C. and 1300 rpm.

### 3. Sample Preparation: Semi-Denaturing SDS-PAGE Gel

After cultivating individual clones from 2. overnight, the OD₆₀₀ of the precultures in 24-cavity plates was adjusted to 0.1, and the samples, 2 ml each, were shaken at 37° C. and 180 rpm for 30 min.

Thereafter, the R-GECO1 was initially induced with 25 mM sodium propionate, pH 8.0, at 30° C. and 180 rpm for 120 min.

Subsequently, the pores were then induced with 0.5 mM IPTG at 30° C. and 180 rpm for 180 min.

### 4. Analysis: Semi-Denaturing SDS-PAGE Gel

After 180 min, 20 μl of each sample was collected and the supernatant was separated from the pellet by centrifugation

The pellet was dissolved in 20 μl resuspension buffer (200 mM Na₃PO₄, 1 mM CaCh) and a further 20 μl 2×SDS buffer was added (total volume 40 μl)

20 μl 2×SDS buffer (+1 mM CaCh) was added to the supernatant (total volume 40 μl)

General information: 12% SDS-PAGE gel, run at 120 V for about 110 min. Gels were recorded using the Amersham Imager600 gel documentation unit (extinction: 520 nm; emission 605/BP40 nm). All gels were exposed identically to guarantee comparability.

### Conclusion:

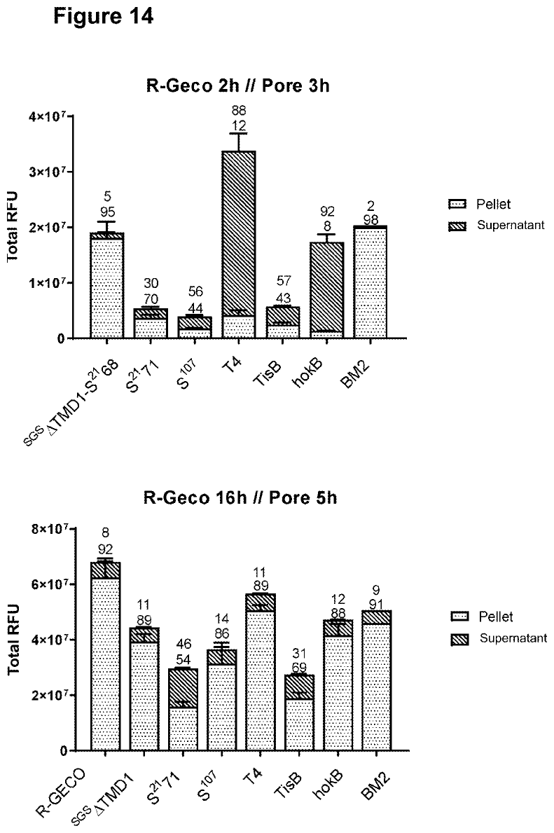

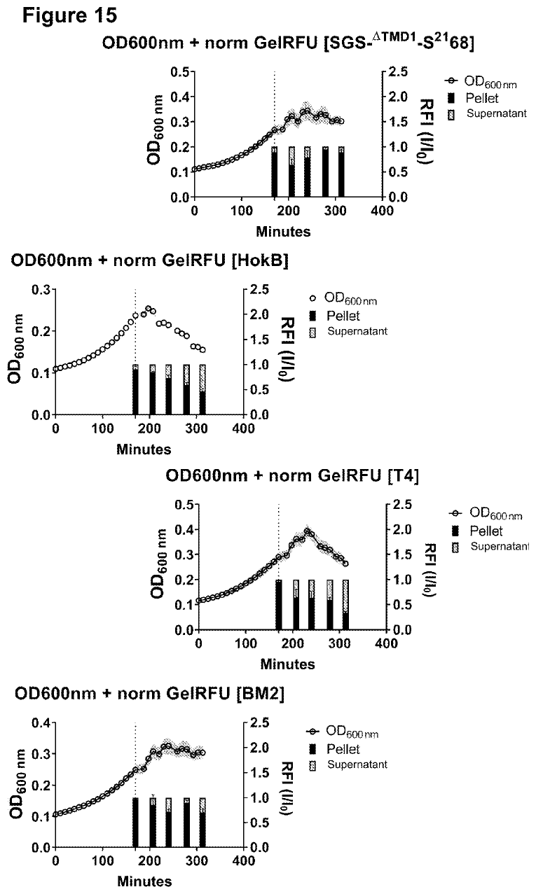

Overall, different pores under the given expression conditions show different propensities to preserve cellular integrity or to lyze the cells. Under exponential growth after 3 h (FIG. 14) and after growth saturation after 16 h, in particular after expression of ^{SGS}ΔTMD1-S²¹68 and BM2, fluorescence is present only in the cell pellet, but not in the extracellular medium (FIG. 15). Specifically, this indicates that the cellular integrity is preserved for these pores. In contrast, the T4 pinholin along with HokB and TisB show distinct cell lysis by an extraordinarily high proportion of fluorescence in the extracellular medium under the exponential growth after 3 h (FIG. 14), but not under saturating growth conditions after 16 h (FIG. 15). This is presumably due to the fact that after growth saturation, the metabolism is reduced to such an extent that insufficient pores can be expressed in order to bring about effective cell lysis.

### Example 6: Assay Validation for the Screening of Pore-Forming Membrane Peptides in Individual Colonies on Agar Plates

### Background:

Alternatively, the newly developed assay can also be applied in colony format on agar plates. In comparison to microtiter plates (see Examples 1-5), screening methods based on colonies enable higher throughput and, moreover, do not rely on expensive laboratory equipment, such as a microtiter plate spectrophotometer. Specifically, there must be a check of the extent to which quantitative analysis or differentiation of pore-forming peptides from non-pore-forming peptides is also possible at the level of individual colonies. The model peptides were, as before, the pore-forming membrane peptide ^{SGS}ΔTMD1-S²¹68 and the non-pore-forming membrane peptide ^{TVMV}ΔTMD1-S²¹68 as positive or negative control.

### Experimental Procedure and Results:

1. Co-Transformation of the R-GECO1 and the Two Pinholin Fragments in Chemically Competent E. coli.

The following combinations were tested:

→BL21(DE3) with pACYCT2-ΔTMD1-S²¹68+pPRO24-R-GECO1
→BL21(DE3) with pACYCT2-^{TVMV}ΔTMD1-S²¹68+pPRO24-R-GECO1

The transformation batches are coated on LB agar with appropriate 25 antibiotics and cultivated overnight at 37° C.

### 2. Procedure the Colony-Based Assay:

On the following day, the colonies are transferred with a filter paper to a 30 further LB agar plate with 25 mM sodium propionate in order to express R-GECO1.

The stamped plate is cultivated for 4 h at 37° C. The expression of the pinholin fragments is subsequently induced with 0.5 mM IPTG

For this purpose, a filter paper is sprayed with the corresponding solution and placed on the colonies for about 10 s

The plate is cultivated for another 2 h at 37° C. until subsequent storage at 4° C. until the following day (with light protection)

On the following day, the fluorescence of the colonies is recorded at the extinction wavelength of 520 nm (0.3 s exposure time)

### 3. Experimental Analysis:

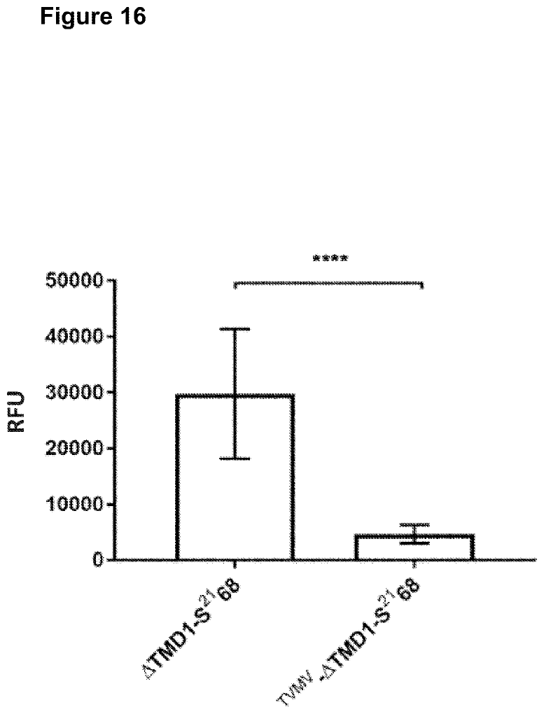

The fluorescence of at least >180 colonies in each case is shown in FIG. 16. The result was a difference of a factor of 6.5 on average between the positive control (ΔTMD1-S²¹68) and negative control (^{TVMV}ΔTMD1-S²¹68).

### Conclusion:

The newly developed method can also be used in the colony format on agar plates to differentiate pore-forming membrane peptides from non-pore-forming membrane peptides. Significant differences in the fluorescent signal between individual colonies can be observed after a single time as a function of the expression of pore-forming ^{SGS}ΔTMD1-S²¹ 68 or non-pore-forming membrane peptide ^{TVMV}ΔTMD1-S²¹68 (FIG. 16).

### Example 7: Model Selection for the Screening of Pore-Forming Membrane Peptides in Colonies on Agar Plates

### Background:

Analogously to the procedure on microtiter plates, there was a check in model selections as to what extent the connection between genotype and phenotype is preserved even in individual colonies on agar plates within the entire selection cycle, and the DNA of a pore-forming membrane peptide can be reidentified from a larger quantity of non-pore-forming membrane peptides, selected and enriched. Analogously to the validation of the method in microtiter plates, screening for colonies enables higher throughput and does not rely on expensive microtiter plate spectrophotometers. The model peptides were, as before, the pore-forming membrane peptide ^{SGS}ΔTMD1-S²¹68 and the non-pore-forming membrane peptide ^{TVMV}ΔTMD1-S²¹68, again as positive or negative control.

### Experimental Procedure and Results:

1. Co-Transformation of the R-GECO1 and the Two Pinholin Fragments in Chemically Competent E. coli.

The following combinations were tested as dilution in each case:

→In the ratio 1:5 of ATMD1-S²¹68 to ^{TVMV}ΔTMD1-S²¹68+R-GECO1
→In the ratio 1:10 of ATMD1-S²¹68 to ^{TVMV}ΔTMD1-S²¹68+R-GECO1

The transformation batches are coated on LB agar with appropriate antibiotics and cultivated overnight at 37° C.

### 2. Procedure for the Colony-Based Assay:

On the following day, the colonies are transferred with a filter paper to a further LB agar plate with 25 mM sodium propionate in order to express R-GECO1

The stamped plate is cultivated for 4 h at 37° C. The expression of the pinholin fragments is subsequently induced with 0.5 mM IPTG. For this purpose, a filter 30 paper is sprayed with the corresponding solution and placed on the colonies for about 10 s.

The plate is cultivated with light protection for another 2 h at 37° C. until subsequent storage at 4° C. until the following day.

On the following day, the fluorescence of the colonies is recorded at the extinction wavelength of 460 nm (0.1 s), 520 nm (1 s) and 630 nm (1 s)

### 3. Analysis:

For the determination of the colonies with the highest fluorescence, only the data from the channel at 520 nm are used.

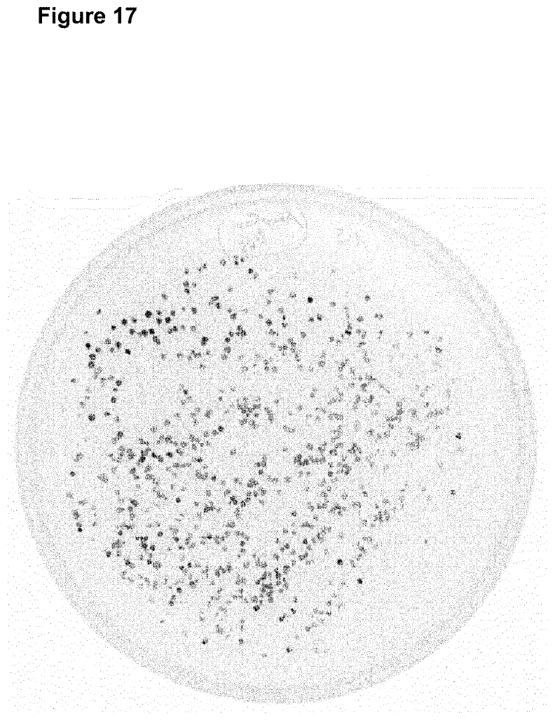

FIG. 17 shows a representative image of the fluorescences of the colonies. 8 colonies of the ratio 1:5 with high fluorescence and 6 colonies of dilution 1:10 were sequenced. Sequencing resulted in the positive control pACYCT2-^{SGS}ΔTMD1-S²¹68 for all colonies.

### Conclusion:

In summary, the pore-forming membrane peptides can be (a) differentiated quantitatively on agar plates from the non-pore-forming membrane peptide ^{TVMV}ΔTMD1-S²¹68 and (b) selected and enriched from a greater dilution of 1:5 and 1:10 (FIG. 17).

### Example 8: Assay Validation at the Single-Cell Level by Means of a Flow Cytometer

### Background:

Alternatively, the newly developed assay can also be used in order to investigate the pore-forming properties or functions of membrane peptides at the single-cell level by means of a flow cytometer. In comparison to microtiter 30 plates (see Examples 1-5) or also colonies on agar plates (see Examples 6 and 7), this has the advantage that the assay can be measured with high resolution and with a high statistical significance: >10⁶ variants within one FACS measurement. Specifically, there must be a check of the extent to which quantitative analysis or differentiation of pore-forming peptides from non-pore-forming peptides is also possible at the level of individual cells. The model peptides were, as before, the pore-forming membrane peptide ^{SGS}ΔTMD1-S²¹68 and the non-pore-forming membrane peptide ^{TVMV}ΔTMD1-S²¹68, again as positive or negative control.

### Experimental Procedure and Results:

### 1. Co-Transformation of the Library in Chemically Competent BL21(DE3)

Transformed individually in each case

→BL21(DE3) with pPRO24-G-GECO1 (reporter)+pTeT7W-^{SGS}ΔTMD1-S²¹68 (positive)
→BL21(DE3) with pPRO24-G-GECO1 (reporter)+pTeT7W-^{TVMV}ΔTMD1-S²¹68 (negative)
→BL21(DE3) with pPRO24-G-GECO1 (reporter)+pTeT7W-^{SGS}ΔTMD1-S²¹68 (positive)+pTeT7W-^{TVMV}ΔTMD1-S²¹68 (negative) in the ratio 1:10 of positive to negative

### 2. Inoculation Overnight:

Colonies of BL21(DE3) on LB agar from 1. were picked and cultivated in 25 culture tubes with 2 ml of LB medium with the necessary antibiotics overnight for about 16 h at 37° C. and 220 rpm.

Mixed variants of 1:10 and 1:100 from positive to negative were not plated, but were transferred directly after transformation into medium and allowed to grow

### 3. FACS Sample Preparation:

After cultivation from 2. overnight, the OD₆₀₀ of the precultures in culture tubes was adjusted to about 0.1, and the samples, 2 ml each, were shaken at 37° C. and 180 rpm for 30 min.

Thereafter, the G-GECO1 was initially induced with 25 mM sodium propionate, pH 8.0, cultivated at 30° C. and 220 rpm for 120 min.

Subsequently, the positive and negative control, as well as the 1:10 and 1:100 mixed cultures were then induced with 0.5 mM IPTG at 30° C. and 220 rpm for 60 min.

After renewed measurement of the OD_{600 nm}, the cultures were washed with 2×PBS, subsequently adjusted with PBS to an OD_{600 nm} of 0.05 and stored on ice

### 4. FACS Measurements

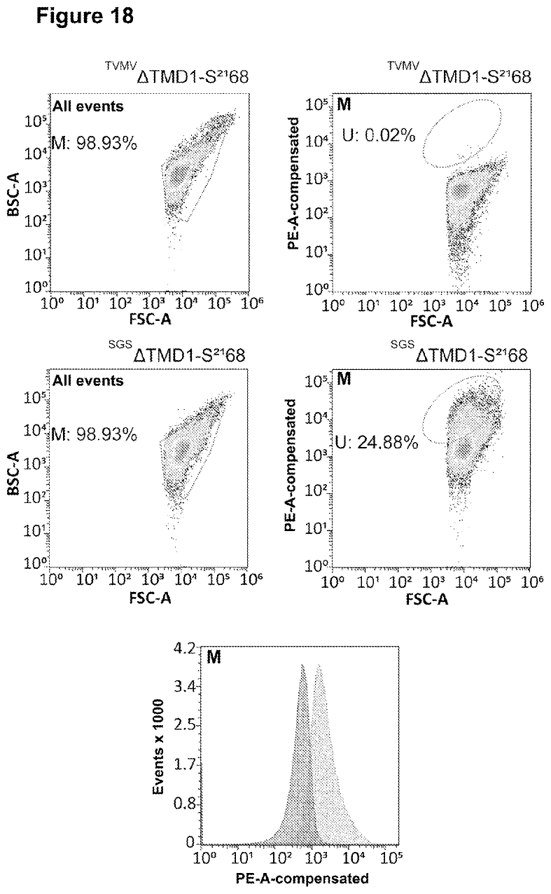

The fluorescence measurements on the FACS (SONY SH800S) were carried out with an extinction of 480 nm and an emission of 525 nm. The assay validation results are summarized in FIG. 18.

### Conclusion:

In summary, in flow-cytometric analysis, two differently fluorescing populations of E. coli result (depending on the expression of a pore-forming ^{SGS}ATMD1-S²¹68 in comparison to a non-pore-forming peptide ^{TVMV}ATMD1-S²¹68). In parallel, a separation into forward and backward scatters indicates that the cellular integrity after expression of the pore-forming peptide is largely preserved and that it is thus possible, i.e., the method is in principle suited, to differentiate membrane peptides having different pore-forming properties at the single-cell level by flow cytometry.

### Example 9: Model Selection at the Single-Cell Level by Means of a Flow Cytometer

### Background:

Alternatively, the newly developed assay can be used to sort and enrich the pore-forming properties or functions of membrane peptides at the level of individual cells by means of a flow cytometer. In comparison to microtiter plates (see Examples 1-5) or also colonies on agar plates (see Examples 6 and 7), this has the advantage that the assay can be carried out quantitatively with a very high throughput, which in principle makes it possible to screen >10⁶ variants within one selection cycle. Specifically, there must be a check of the extent to which quantitative analysis or differentiation of pore-forming from non-pore-forming peptides is also possible at the level of individual cells. For this purpose, the already known transmembrane peptides ^{SGS}ΔTMD1-S²¹68 and ^{TVMV}ΔTMD1-S²¹68 served as positive or negative control.

### Experimental Procedure and Results:

### 1. Co-Transformation of the Peptides in Chemically Competent BL21(DE3)

Transformed individually in each case

→BL21(DE3) with pPRO24-G-GECO1 (reporter)+pCTRL2-^{SGS}ΔTMD1-S²¹68 (positive)
→BL21(DE3) with pPRO24-G-GECO1 (reporter)+pCTRL2-^{TVMV}ΔTMD1-S²¹68 (negative)
→BL21(DE3) with pPRO24-G-GECO1 (reporter)+pCTRL2-^{SGS}ΔTMD1-S²¹68 (positive)+pCTRL2-^{TVMV}ΔTMD1-S²¹68 (negative) in the ratio 1:10 of positive to negative

### 2. Inoculation Overnight:

Colonies of BL21(DE3) on LB agar from 1. were picked and cultivated in culture tubes with 2 ml of LB medium with the necessary antibiotics overnight for about 16 h at 37° C. and 220 rpm.

Mixed variants of 1:10 and 1:100 from positive to negative were not plated, but were transferred directly after transformation into medium and allowed to grow

### 3. FACS Sample Preparation:

After cultivation from 2. overnight, the OD₆₀₀ of the precultures in culture tubes was adjusted to about 0.1, and the samples, 2 ml each, were shaken at 37° C. and 180 rpm for 30 min.

Thereafter, the G-GECO1 was initially induced with 25 mM sodium propionate, pH 8.0, cultivated at 30° C. and 220 rpm for 120 min.

Subsequently, the positive and negative control, as well as the 1:10 mixed cultures were then induced with 0.5 mM IPTG at 30° C. and 220 rpm for 60 min.

After renewed measurement of the OD_{600 nm}, the cultures were washed with 2×PBS, subsequently adjusted with PBS to an OD_{600 nm} of 0.05 and stored on ice

### 4. FACS Measurements

The fluorescence measurements on the FACS (SONY SH800S) were carried out with an extinction of 480 nm and an emission of 525 nm. The results of the model selection experiments on the FACS are summarized in FIG. 18.

### Conclusion:

In summary, the pore-forming membrane peptide ^{SGS}ΔTMD1-S²¹68 can 30 be (a) differentiated quantitatively at the single-cell level from the non-pore-forming membrane peptide ^{TVMV}ΔTMD1-S²¹68 and (b) selected and enriched from a greater dilution of 1:10. It should be noted that depending on the expression of a pore-forming peptide, in this case ^{SGS}ΔTMD1-S²¹68, the cell integrity of the cell is preserved to the extent that selection by FACS is possible.

### Example 10: Validation of a Method for Screening Molecular Sensors and Switches

### Background:

In addition to the development of a method for screening pore-forming membrane peptides, nanopores and ion channels (see Examples 1-9), the method can be reconfigured in order to optimize molecular sensors and switches in combination. Specifically, the challenge of developing methods for screening protein sensors or switches is to measure the molecular state of a sensor in each case with and without a ligand (and thus to select for the highest possible differential in the signal). It is critical and technically demanding to supply a defined amount of a ligand to the sensor or switch in order to activate the latter.

For this purpose, the sensor or switch can be exported extracellularly or periplasmically, for example. In these cases, an externally added ligand can associate with the sensor and switch comparatively easily. This also applies in the case of periplasmically exported sensors and switches since the outer E. coli membrane is comparatively permeable to a large number of low-molecular substances. However, this is only possible to a limited extent for fluorescent protein sensors consisting of a plurality of independently folding domains and also limits the folding properties and stability of the sensor or switch. Alternatively, in the case of cytosolic expression, the challenge is to allow the ligands to flow into or out of the cell via the membrane. Due to the intrinsically impermeable membrane, this strongly limits the number and nature of the ligands, however.

As part of the underlying method, the expression of nanopores is now to enable an increased permeability of the inner E. coli membrane via the expression of nanopores. The key development step is to investigate the extent to which low-molecular substances that are many times larger than Ca²⁺ ions can diffuse through nanopores of different sizes. Specifically, the R- and G-GECOs are to be replaced by L-Glu-specific fluorescence sensors, so-called iGluSnFRs, in order to detect the inflow and outflow of L-Glu beyond Ca²⁺ ions.

### Experimental Procedure and Results:

1. Co-Transformation of iGluSNFr and the Two Pinholin Fragments in Chemically Competent E. coli.

Transformed individually in each case:

→BL21(DE3) with pPRO24-iGluSNFr (reporter)+pCTRL2-^{SGS}ΔTMD1-S²¹68 (positive)
→BL21(DE3) with pPRO24-iGluSNFr (reporter)+pCTRL2-^{TVMV}ΔTMD1-S²¹68 (negative)
→BL21(DE3) with pPRO24-iGluSNFr (reporter)+pCTRL2-BM2 (negative)
→BL21(DE3) with pPRO24-iGluSNFr (reporter)+pCTRL2-S²¹71-M4A
→BL21(DE3) with pPRO24-iGluSNFr (reporter)+pCTRL2-S²¹71-M3A
→BL21(DE3) with pPRO24-iGluSNFr (reporter)+pCTRL2-HokB
→BL21(DE3) with pPRO24-iGluSNFr (reporter)+pCTRL2-K_{CV}^{NTS}
→BL21(DE3) with pPRO24-iGluSNFr (reporter)+pCTRL2-K_{CV}^{G77S}
→BL21(DE3) with pPRO24-iGluSNFr (reporter)+pCTRL2-K_{PBCV1}
→BL21(DE3) with pPRO24-iGluSNFr (reporter)

### 2. Inoculation Overnight:

Colonies of BL21(DE3) on LB agar from 1. were picked and cultivated in deep well plates with 300 μl of M9 medium (defined medium without glutamate) with the necessary antibiotics overnight for about 16 h at 37° C. and 1300 rpm.

### 3. Microtiter Plate Readout:

After cultivating individual clones from 2. overnight, the OD₆₀₀ of the precultures in microtiter plates (MTP) was adjusted to 0.1, and the samples, 200 μL M9 medium each, were shaken at 37° C. and 180 rpm for 30 min.

Thereafter, the iGluSNFr was initially induced with 25 mM sodium propionate, pH 8.0, and the fluorescence was measured over 290 min.

Subsequently, the two pore-forming peptides were then induced with 0.5 mM IPTG, and the fluorescence was measured over another 260 min.

General information: The measurement in the reader is carried out at 30° C. every 10 min. The plate is shaken at 180 rpm between the measurements. The OD_{600 nm} and the fluorescence (Ex: 480 nm; Em: 525 nm) are measured. The volume in the cavities is 200 μl after addition of all inductors.

### Conclusion:

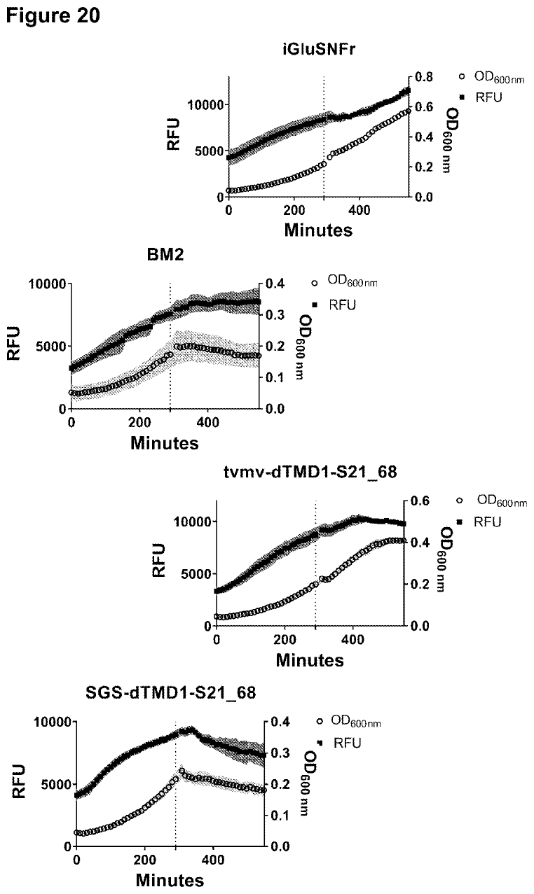

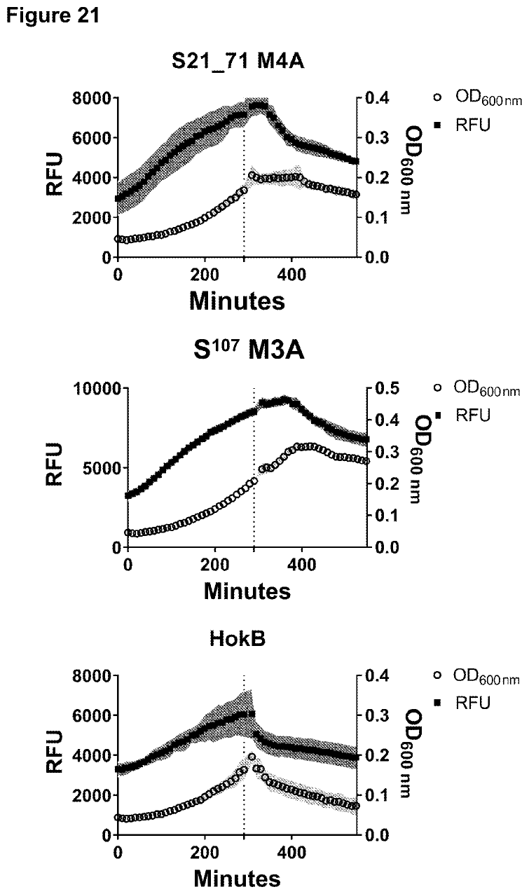

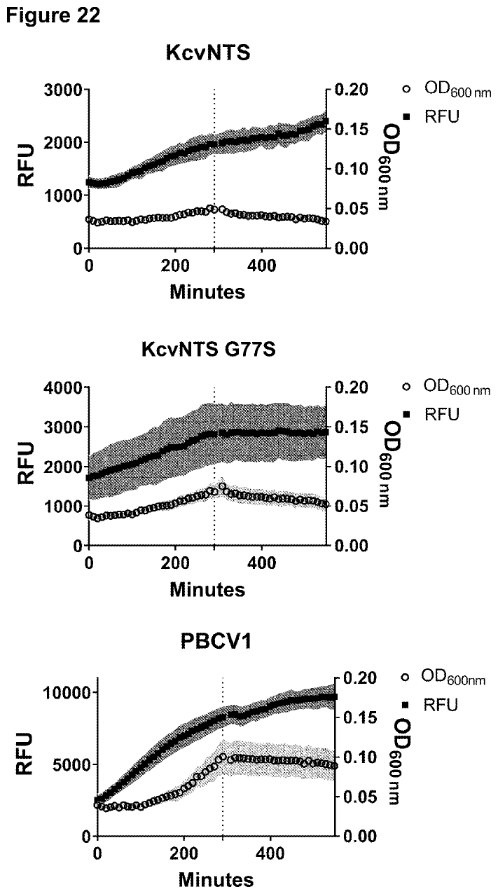

Overall, the expression of the pore-forming membrane peptide ^{SGS}ΔTMD1-S²¹68 results in a reduction of the fluorescent signal of the iGluSNFr; in contrast, after expression of the non-pore-forming membrane peptide ^{TVMV}ΔTMD1-S²¹68, the iGluSNFr-related signal remains unchangedly high (results combined in each case in FIG. 20). This is due to the fact that L-Glu from the cell can flow out of the cell after co-expression of the pore-forming membrane peptide ^{SGS}ΔTMD1-S²¹68. The same applies to the pore-forming membrane peptides S²¹-71 M4A, S-107 M3A and HokB, which result in a strong reduction of the iGluSNFr dependent signal (see FIG. 21). Expression of the various negative controls, such as the proton channel BM2 and the K⁺-permeable channels K_{CV}^{NTS}, K_{CV}^{NTS} G77S, and K_{PBCV1} result in each case in cell death, but not in a reduction in fluorescence (see FIG. 20 and FIG. 22). In summary, this shows that, after expression of sufficiently large nanopores in the inner membrane, an outflow of L-Glu from the cell occurs, which in turn can be detected indirectly via the decrease in the fluorescent signal.

### Example 11—Validation of the Method in a Microfluidic Experiment Arrangement

### Background:

Microfluidic methods enable high-resolution functional studies, in particular in combination with optical measuring methods, for example based on fluorescence. Microfluidic methods offer a number of technical advantages, such as miniaturization of bioanalytical methods (Journal of Laboratory Automation, 2013, 18, 350-66), temporally resolved dynamic studies of microorganisms at the single-cell level in a microfluidic cultivation chamber (see examples in the respective review articles, Journal of Molecular Biology, 431, 2019, 4569-4588) or possibilities for screening in high throughput in droplet-based compartments with individual cells or in microcolonies (see examples in the respective review articles) Current Opinion Structural Biology, 2018, 48, 149-156, and Current Opinion in Chemical Biology, 2017, 37, 137-144 and specifically ACS Synthetic Biology, 2017, 6, 1988-1995).

In this context, the pore-forming properties of two different membrane proteins (specifically of the S²¹68 pinholin and T4 holin) were investigated in a microfluidic cultivation chamber on a single-cell level in temporally resolved form with a fluorescence microscope. In addition and as a continuation of Example 5, there was also an investigation as to what extent the expression of a pore-forming membrane protein lyzes the cells or to what extent the cellular integrity in the form of a shell is preserved.

### Experimental Procedure and Results:

1. Co-Transformation of G-GECO1 and the Nanopore Variants in Chemically Competent E. coli.

Transformed individually in each case:

→BL21(DE3) with pPRO24-G-GECO1 (reporter)+pCTRL2.T7.100.sRBS-S²¹68
→BL21(DE3) with pPRO24-G-GECO1 (reporter)+pCTRL2.T7.100.T4

### 2. Inoculation Overnight:

Colonies of BL21(DE3) on LB agar plates from 1. were picked and cultivated in culture tubes with 2 ml of LB medium and the necessary antibiotics overnight for about 16 h at 37° C. and 180 rpm.

### 3. Microfluidics Readout:

After the cultivation of individual clones from 2. overnight, the cells were transferred to fresh sterile-filtered LB medium, the OD₆₀₀ of the precultures was 20 adjusted to 0.1, and subsequently grown at 37° C. for a period of 3 h up to an OD₆₀₀ of 2.

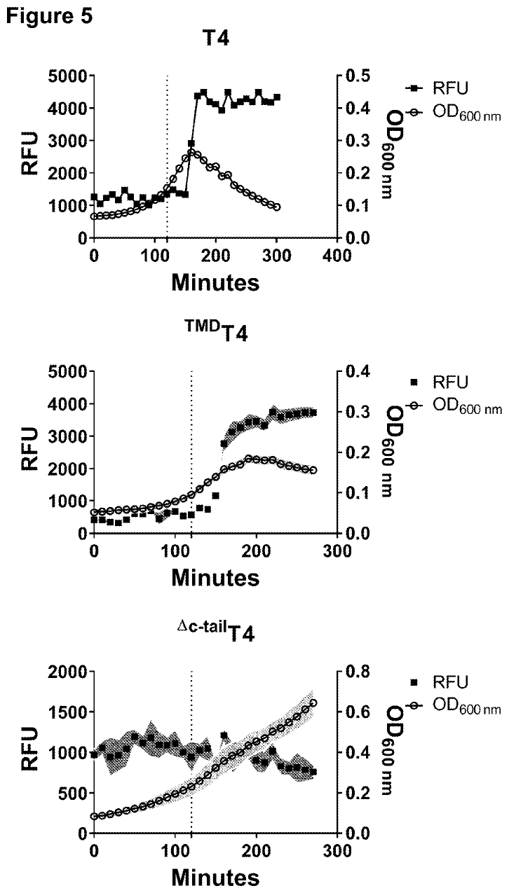

For the cultivation and functional fluorescence-microscopic examination of the cells in a microfluidic cultivation chamber, it was possible to use a previously published microfluidic chip design (see Supplementary FIG. 5 in Nature Communications, 2020, 11, 2746).

After the microfluidic chip was flooded with LB medium, the cells were added and the flow rate through the microfluidic chip was adjusted to 1 μl/min. The cells were left in the microfluidic chip at 37° C. for 3 h, so that they were able to collect and grow accordingly in the cultivation chambers.

Thereafter, the expression of G-GECO1 was induced by a change to LB medium with 25 mM sodium propionate, pH 8.0, at 30° C. for 2 h.

Subsequently, the nanopores were then induced by a change to LB medium with 0.5 mM IPTG, and the development of the fluorescent signal was measured over 16 h.

The fluorescent signal was measured by means of a fluorescence microscope at 30° C. in intervals of 3 minutes. A transmitted-light image and then a fluorescence image (Ex: 488 nm; Em: 504-539 nm) in the form of a Z-stack were taken each time. From the induction of G-GECO1, a flow rate of 4 μl/min was used.

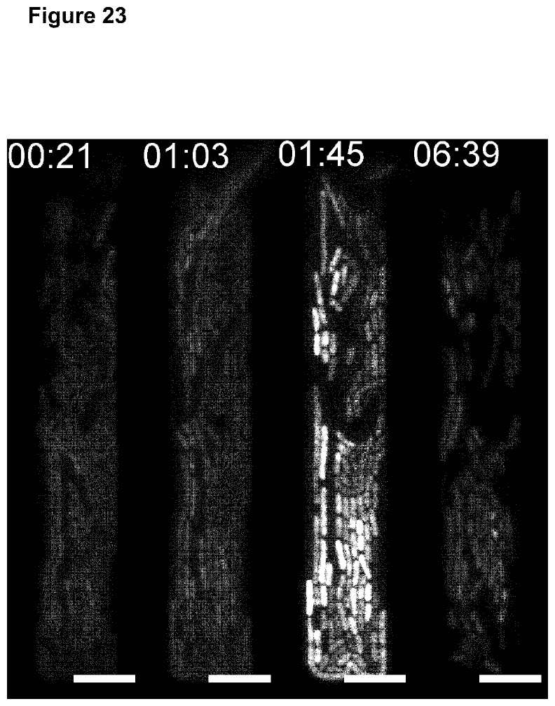

Image sections of the fluorescence-microscopic images in the microfluidic cultivation chamber are shown by way of example for the S²¹68 pinholin at four different points in time (FIG. 23). The induction of the expression of the nanopore with IPTG was carried out in the image at the 21 min time (indicated in FIG. 23 by 0:21).

### 4. Experimental Analysis:

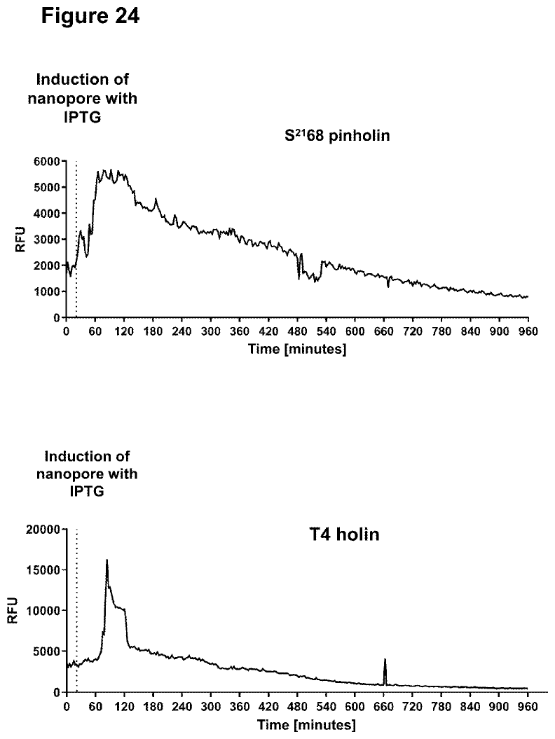

For the analysis, the entire cells from a single cultivation chamber (shown by way of example in FIG. 23) were analyzed with the aid of image analysis software (Fiji ImageJ 1.51; Nature Methods, 2012, 9, 676-82) and the fluorescence in relative fluorescence units (RFU) was plotted against the time (FIG. 24). Based on the curve of the fluorescent signal, detailed statements about the cell integrity or the outflow and the bleaching behavior of the sensor can then be made.

### Conclusions:

In summary, the test series demonstrates the application of the method in a microfluidic device. Specifically, cells were cultivated in a microfluidic cultivation chamber, and the pore-forming properties of the S²¹68 pinholin and the T4 holin were investigated at the single-cell level in temporally resolved form under a fluorescence microscopy.

As a function of different membrane proteins, different fluorescent signals resulted: The expression of the S²¹68 pinholin causes the cell division to stop quickly and the fluorescent signal to increase quickly (FIG. 24). However, the cellular integrity measured at the form of the cells remains intact over a comparatively long period of time (FIG. 23). The expression of the T4 holin also likewise causes the cell division to stop quickly and the fluorescent signal to increase quickly. In the case of the T4 holin, however, the fluorescent signal rapidly decreases again (within 30 min). This is due to the fact that the T4 holin is likely to very quickly form very large pores, and these pores result in rapid inflow of Ca²⁺ and thus glowing of the cell, before outflow of the sensor or loss of cellular integrity and cell lysis then occur comparatively quickly (FIG. 24).

In addition to Example 5, the results achieved show that the “cellular container” remains intact despite pore-forming properties, and inflow of Ca²⁺ ions into the cell occurs as a function of the nanopore. For comparatively large nanopores, such as the T4 holin, the time window is comparatively short (in this case 30 min) before either a loss of cellular integrity or outflow of the sensor occurs (FIG. 24).

### Example 12—Selection Experiments for Finding New Properties and Functions of Pore-Forming Membrane Peptides in Microtiter Plates

### Background:

In addition to model selections, a library of pore-forming variants of the S²¹71 pinholin in the microtiter plate format was also screened for changed pore-forming properties. The primary goal of a real selection is to perform a check of the extent to which the method can be used to select membrane proteins with novel pore-forming properties and functions.

Specifically, the three N-terminal amino acids of the S²¹71 pinholin were randomized by means of saturation mutagenesis in order to check which mutations have a positive or negative effect on the pore-forming properties of the wild-type S²¹71 pinholin. In addition, there should be a clarification of the extent to which the promoter strength, measured at the half-maximum time c, and the slope d have an influence on the dynamic range of the method in the context of a library selection.

### Experimental Procedure and Results:

### 1. Design of the Mutagenesis Primer:

The mutagenesis primer for the S²¹71 pinholin designed for the following DNA sequence or amino acid sequence:

$$N - terminal sequence of the {S}^{21} 71 pinholin$$

$$\begin{matrix}DNA sequence & ATG & AAA & TCT & ATG & GAC & AAA & ATC & TCA & ACT \\ Protein sequence & M & K & S & M & D & K & I & S & T\end{matrix}$$

$$Primer ( SEQ ID NO : 22 )$$

$$\begin{matrix}{5}^{′} - TGATGAGG & CAT & ATG & NNK & NNK & NNK & GAC & AAA & ATC & TCA & ACT - {3}^{′} \\ Protein sequence & M & X 1 & X 2 & X 3 & D & K & I & S & T & \end{matrix}$$

The randomized codons in the context of the N-terminally randomized pinholin library ^{3×NNK}S²¹68 are in each case denoted by X₁, X₂ and X₃ or position 1, position 2 and position 3.

### 2. Cloning of the Library

Initially, the S²¹71 pinholin was amplified by the above-mentioned forward primer and a suitable reverse primer and cloned with the respective interfaces for NdeI/KpnI into the pCTRL2 or pCTRL2.T7.100.sRBS vector.

A library each was built from about 300,000 clones for pCTRL2 and 270,000 clones for pCTRL2.T7.100.sRBS.

3. Co-transformation of G-GECO1 with the library and control reference in chemically competent BL21(DE3)

Transformed individually in each case:

→BL21(DE3) with pPRO24-G-GECO1 (reporter)+pCTRL2.T7.100.sRBS-^{3×NNK}S²¹68
→BL21(DE3) with pPRO24-G-GECO1 (reporter)+pCTRL2-^{3×NNK}S²¹68
→BL21(DE3) with pPRO24-G-GECO1 (reporter)+pCTRL2.T7.100.sRBS-S²¹71 M4A
→BL21(DE3) with pPRO24-G-GECO1 (reporter)+pCTRL2-S²¹71 M4A

### 4. Inoculation Overnight:

Colonies of BL21(DE3) on LB agar from 3. were picked and cultivated in deep well plates with 300 μl of LB medium with the necessary antibiotics overnight for about 16 h at 37° C. and 1300 rpm.

### 5. Microtiter Plate Readout:

After cultivating individual clones from 4. overnight, the OD₆₀₀ of the precultures in microtiter plates was adjusted to 0.1, and the samples, 200 μL each, were shaken at 37° C. and 180 rpm for 30 min. Within the framework of the pCTRL2-^{3×NNK}S²¹68 library, 271 clones were thus picked. Within the framework of the pCTRL2.T7.100.sRBS-^{3×NNK}S²¹68 library, 576 clones were picked.

Thereafter, the G-GECO1 was initially induced with 25 mM sodium propionate, pH 8.0, and the fluorescence was measured over 110 min.

Subsequently, all nanopore variants were then induced with 0.5 mM IPTG, and the fluorescence was measured over another 190 min.

General information: The measurement in the reader is carried out at 30° C. every 3 min. The plate is shaken at 180 rpm between the measurements. The OD₆₀₀ and the fluorescence (Ex: 485 nm; Em: 525 nm) are measured. The volume in the cavities is 200 μl after addition of all inductors.

### 6. Analysis

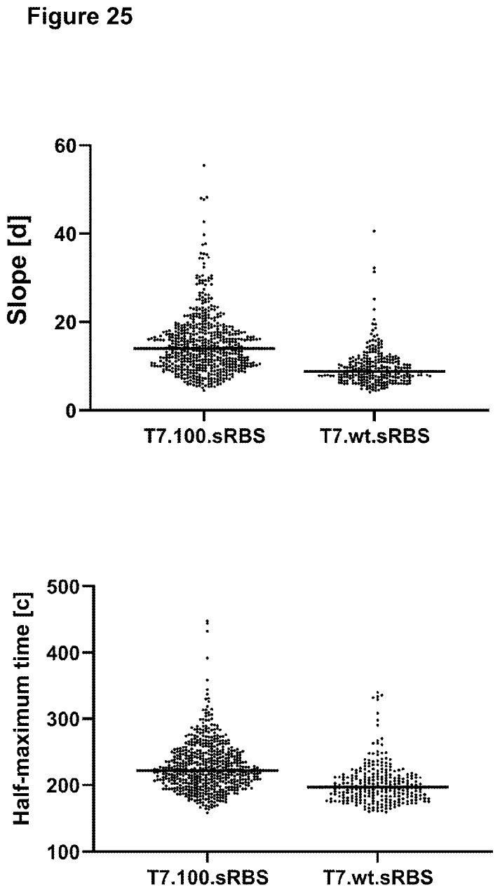

For the analysis, the curves were fitted empirically with equation 1 in order to quantitatively determine the half-maximum time c or the slope d, for example. The distribution of the parameters investigated with respect to the half-maximum time c and the slope d of the two libraries pCTRL2-^{3×NNK}S²¹68 and pCTRL2.T7.100.sRBS-^{3×NNK}S²¹68 is summarized in FIG. 25.

Individual mutants from the pCTRL2.T7.100.sRBS-^{3×NNK}S²¹68 library were subsequently sequenced (240 sequenced of the total of 576 measured) and subjected to a detailed sequence structure function analysis. Sequences with methionine were not taken into account here due to the possibility of an alternatively start codon. Sequences with premature stop codons were also not taken into account. Sequences with cysteines were also not taken into account due to possible disulfide bridges. The same applies to prolines, which only rarely occurred. In addition, sequences with reading-frame shift were also not taken into account. This ultimately resulted in a selection of 142 sequences of originally 240 sequenced variants, which were respectively analyzed.

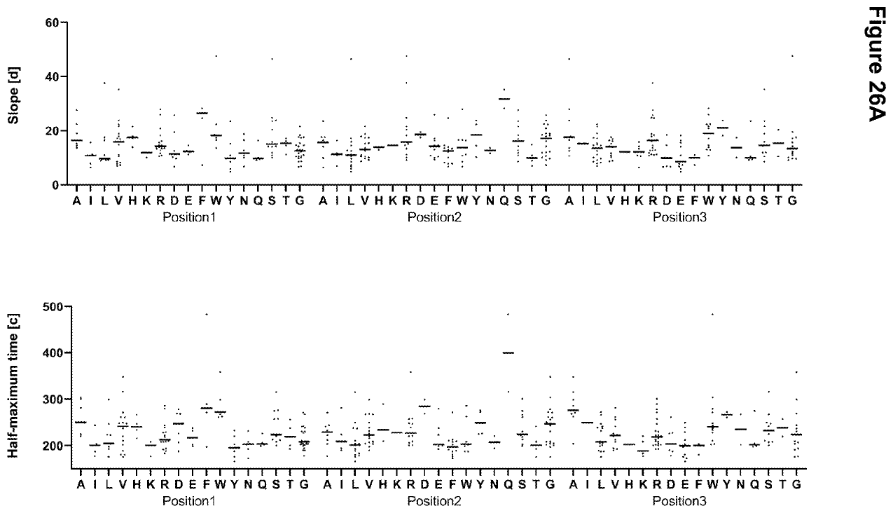

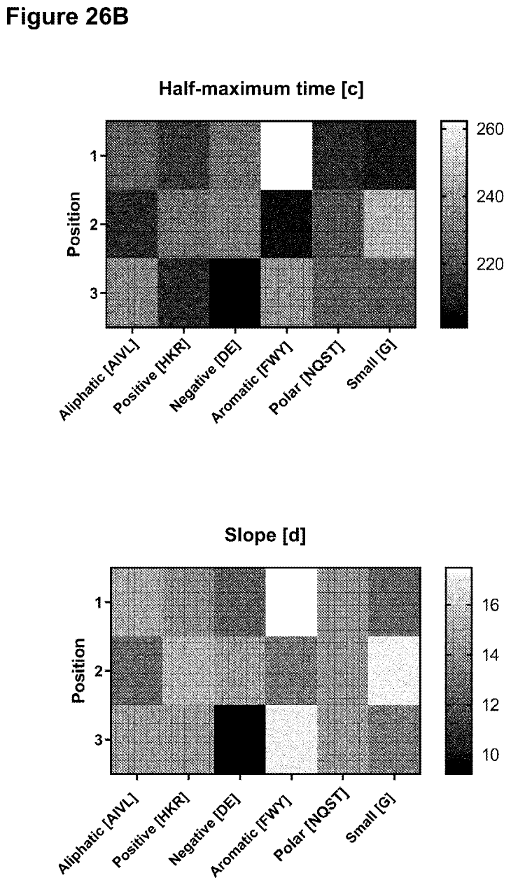

For the analysis of the underlying structure-function properties, the average half-maximum time c and the average slope d were calculated in each case for each individual amino acid at the three different positions X₁, x₂ and X₃ (FIG. 26A). The average values were calculated independently of the context of the sequence.

In parallel, the individual amino acids were combined into groups with comparable chemical properties, and the average half-maximum time c and the average slope d were calculated in each case. These results are summarized as a heat map (FIG. 26B). The average values were calculated independently of the context of the sequence.

In addition, specific sequences with characteristic pore-forming properties (in each case positive and negative) together with the values for the respective half-maximum time c and the slope d are summarized in Table 1.

### Conclusion:

In addition to Example 4, within the scope of a screening campaign with up to 8,000 randomized variants, the influence of the N-terminal sequence of the S²¹-71 pinholin on its pore-forming properties was systematically investigated.

Initially, with the aid of the weaker promoters (specifically, T7.100.sRBS in contrast to T7.wt.sRBS), the pore-forming properties of the ^{3×NNK}S²¹68 library were better experimentally resolved than with the stronger promoter (FIG. 25). Specifically, this becomes clearly apparent due to a greater scattering from the average value with respect to the slope and the half-maximum time and consequently improves the dynamic range of the method so that, due to the weaker protein expression, stronger pore images can be better differentiated in each case.

In addition, a sequence-structure-function analysis shows that negative charges at position 3 measured at the half-maximum time c accelerate pore formation by way of example (FIG. 26B, Table 1). In contrast, aromatic amino acids at positions 1 and 3 measured at the half-maximum time may inhibit pore formation (FIG. 26B, Table 1).

TABLE 1
Singularized clones from the screening
of the ^{3xNNK}S21-68 pinholin library

| | N-terminal | Half-maximum | | |
|---|---|---|---|---|
| Clone ID | sequence | time [c] | Slope [d] | Comment |
| P14E5 | YLE | 166.1 | 4.908 | Accelerating |
| P14C11 | NFE | 172.3 | 6.835 | Accelerating |
| P15D3 | DWD | 187.4 | 6.727 | Accelerating |
| P7D10 | AGW | 304.1 | 22.49 | Inhibitory |
| P3G3 | WRG | 358.7 | 47.62 | Inhibitory |
| P3E5 | FQW | 483.1 | 28.25 | Inhibitory |

Note:
The complete sequences of the following clones are summarized in the sequence listing under SEQ ID NOs: 44-49.

### Example 13—Selection Experiments for Finding New Properties and Functions of Pore-Forming Membrane Peptides in Microtiter Plates III

### Background:

In addition to the model selections (see Example 3) and the screening of the S²¹68 pinholin library with up to 8,000 different variants (see Example 4 and 12), the S²¹ 68 pinholin was additionally truncated in increments of two amino acids and subjected to a functional investigations with the aid of the method.

On the one hand, there must be a check of the extent to which conclusive structure-function properties can be identified with the aid of the method in the context of a systematic screening. On the other hand, there must be a check of which components are sufficient and necessary for the pore formation of the S²¹68 pinholin. Such minimal nanopore motifs then offer, by way of example, promising starting points for further construction projects. In addition, there must be a check of the extent to which course of the fluorescent signal is comparable for different screening formats, specifically in microtiter plates and on the basis of individual colonies on agar plates.

### Experimental Procedure and Results:

### Variant 1: Screening of the S²¹68 Truncation Library in Microtiter Plates

1. Co-Transformation of G-GECO1 and the Truncated S²¹68 Pinholin (SEQ ID NOs: 28-42) in Chemically Competent E. coli.

Transformed individually in each case:

→BL21(DE3) with pPRO24-G-GECO1 (reporter)+pCTRL2.T7.100.sRBS-S²¹68 truncation library
→BL21(DE3) with pPRO24-G-GECO1 (reporter)+pCTRL2.T7.100.sRBS-S²¹71
→BL21(DE3) with pPRO24-G-GECO1 (reporter)+pCTRL2.T7.100.sRBS-S²¹71 M4A

### 2. Inoculation Overnight:

Colonies of BL21(DE3) on LB agar from 1. were picked and cultivated in deep well plates with 300 μl of LB medium with the necessary antibiotics overnight for about 16 h at 37° C. and 1300 rpm.

### 3. Microtiter Plate Readout:

After cultivating individual clones from 2. overnight, the OD₆₀₀ of the precultures in microtiter plates was adjusted to 0.1, and the samples, 200 μL each, were shaken at 37° C. and 180 rpm for 30 min.

Thereafter, the G-GECO1 was initially induced with 25 mM sodium propionate, pH 8.0, and the course of the fluorescent signal was measured over 110 min.

Subsequently, the expression of the S²¹68 truncation variant was then induced with 0.5 mM IPTG, and the course of the fluorescent signal was measured over another 190 min.

General information: The measurement in the microtiter plate spectrometer was carried out at 30° C. at intervals of 3 min. The microtiter was shaken at 180 rpm between the measurements. The OD₆₀₀ and the fluorescence (Ex: 485 nm; Em: 525 nm) are measured. The volume in the cavities is 200 μl after addition of all inductors.

### 4. Experimental Analysis:

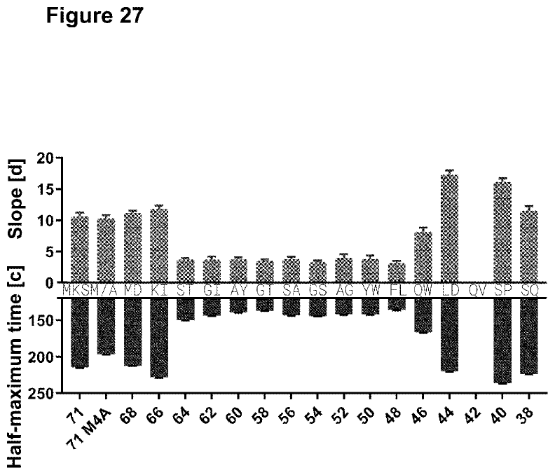

For the analysis, the curves were fitted empirically with equation 1 in order to quantitatively determine the half-maximum time c or the slope d. The distribution of the parameters investigated with respect to the half-maximum time c and the slope d of the truncation is summarized in (FIG. 27).

### Variant 2: Screening of the S²¹68 Truncation Library in Agar Plates

1. Co-Transformation of G-GECO1 and the Truncated S²¹68 Pinholin (SEQ ID NOs: 28-42) in Chemically Competent E. coli.

Transformed individually in each case:

→BL21(DE3) with pPRO24-G-GECO1 (reporter)+pCTRL2.T7.100.sRBS-S²¹68 truncation library.

### 2. Growth Overnight:

Colonies of BL21(DE3) were spread onto LB agar and grown overnight for about 16 hours at 37° C.

### 3. Agar Plate Readout:

At the beginning, the reporter G-GECO1 was induced by spraying 500 mM sodium propionate, pH 8.0, onto the agar plate using an airbrush, so that all colonies were slightly wetted. The agar plates were then incubated in the dark at 20° C. for 180 minutes.

Thereafter, the expression of the truncation variants was induced by spraying 50 mM IPTG solution onto the agar plate, again using an airbrush and slightly wetting all colonies. The agar plates were then incubated in the dark at 20° C. for another 240 minutes.

From the time of IPTG addition, images were recorded at a distance of one 15 hour starting at the time 0 minutes to 240 minutes

General information: The measurement is carried out in blue light in a gel documentation device (Ex: 470 nm; Em: F-590.M58 UV filter). All plates were recorded with the same exposure time to guarantee comparability.

### 4. Experimental Analysis:

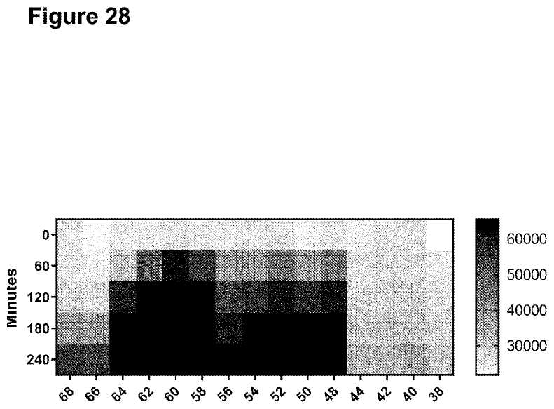

For the analysis, the data of one image detail each were analyzed with the aid of image analysis software (Fiji ImageJ 1.51; Nature Methods, 2012, 9, 676-82), and the maximum fluorescence was plotted against the time (FIG. 28).

### Conclusion:

Specifically, a truncation of the S²-68 pinholin in increments of two 30 amino acids causes different fluorescent signals and thereby provide new insights into the structure-function properties, which are the basis for the pore formation of the S²¹-68 pinholin. Measured at the half-maximum time c and the slope d, the two N-terminal amino acids D and K in the context of S²¹-68 pinholin have an inhibitory effect on the formation of the nanopore. A further truncation improves the pore-forming properties until the complete truncation of the aromatic motif YWFQLW. By way of example, the S²-48 pinholin variant can now be used as a minimal nanopore motif for further construction projects.

With regard to the different screening formats, the course of the fluorescent signals in microtiter plates (quantified and summarized in FIG. 27) is consistent with the fluorescent signals in the colony format on agar plates (FIG. 28). The latter thus enables low-cost screenings in colonies on agar plates and can be used by way of example to preselect libraries with a very high throughput, in order to then subsequently quantify the pore-forming properties for a reduced number of variants with the aid of a fluorescence spectrometer with respect to the half-maximum time c and the slope d.

1. A screening method comprising the steps of:
a) providing a cell and a medium surrounding the cell,
wherein the cell has a cell membrane impermeable to a reporter substance,
wherein the quotient of the concentration of the reporter substance in the interior of the cell and the concentration of the reporter substance in the medium surrounding the cell is at least 2 or at most 0.5;
b) detecting a signal that is dependent on the concentration of the reporter substance in the cell and that is generated with the aid of a sensor protein expressed in the cell;
c) inducing the expression of a membrane protein that increases the permeability of the cell membrane to the reporter substance;
d) detecting the signal that is dependent on the concentration of the reporter substance in the cell and that is generated with the aid of the sensor protein expressed in the cell;
e) calculating one or more comparative parameters from the signal strengths detected in steps b) and d), wherein step b) is carried out before step c) and wherein step d) is carried out after step c),
wherein steps a) to e) are carried out for at least two membrane proteins having different amino acid sequences and/or for at least two sensor proteins having different amino acid sequences.

2. The screening method according to claim 1, comprising the further step of:
f) determining the DNA sequence encoding the membrane protein and/or the DNA sequence encoding the sensor protein.

3. The screening method according to claim 1, wherein the cell is a microorganism.

4. The screening method according to claim 1, wherein the cell is Escherichia coli, and the cell membrane is the inner membrane of E. coli.

5. The screening method according to claim 2, wherein steps a) to f) are carried out for at least 5 membrane proteins with different amino acid sequences.

6. The screening method according to at least claim 2, wherein steps a) to f) are carried out for at least 5 sensor proteins with different amino acid sequences.

7. The screening method according to claim 1, wherein the signal is selected from one or more of a fluorescence signal, a bioluminescence signal, and an absorption signal.

8. The screening method according to claim 1, wherein the sensor protein is selected from the group of Ca²⁺-specific protein sensors and L-Glu-specific protein sensors.

9. The screening method according to claim 1, wherein the reporter substance is selected from the group consisting of cations and amino acids.

10. The screening method according to claim 1, wherein the membrane protein is selected from the group consisting of pore-forming membrane proteins and membrane transporters.

11. The screening method according to claim 1, wherein the membrane protein is selected from the group consisting of holins, pinholins, and ion channels.

12. The screening method according to claim 1, wherein the membrane protein is selected from the group consisting of S²¹-68 (SEQ ID NO:1), S²¹-71 (SEQ ID NO:2), S²¹-71 M4A (SEQ ID NO:3), ^{SGS}ΔTMD1S²¹68 (SEQ ID NO:4), ^{TVMV}ΔTMD1-S²¹68 (SEQ ID NO:5), S¹⁰⁵ (SEQ ID NO:6), S¹⁰⁷ (SEQ ID NO:7), S¹⁰⁷-M3A (SEQ ID NO:8), T4 pinholin (SEQ ID NO:9), T4^{ΔC-Tail} (SEQ ID NO:10), ^{ΔN-Tail}T4^{ΔC-Tail} (SEQ ID NO:11), K_{CV}^{NTS} (SEQ ID NO: 2), K_{CV}^{NTS G77S} (SEQ ID NO:13), K_{CV}^{PBCV1} (SEQ ID NO:14), HokB (SEQ ID NO:15), TisB (SEQ ID NO:16), αHLA (SEQ ID NO:17), cWZA (SEQ ID NO:18), BM2 (SEQ ID NO:19), HCV TME1 (SEQ ID NQ:20), HCV TME2 (SEQ ID NO:21) and variants thereof having sequence identity to at least one of the sequences of SEQ ID NO: 1-21 of at least 90%.

13. The screening method according to claim 1, wherein step d) is carried out at most 120 minutes after step c).

14. The screening method according to claim 1, wherein step d) is carried out at least five times, and the calculation of the comparative parameter from the signal strengths according to step e) includes empirically fitting the data obtained on the signal strengths with a sigmoid function, wherein the comparative parameter calculated from the signal strengths is the half-maximum time c and/or the maximum slope d.

15. The screening method according to claim 14, wherein the data obtained on the signal strengths are fitted empirically with the following equation 1
$$f ( x ) = a + \frac{b}{1 + {e}^{\frac{- ( x - c )}{d}}} \qquad Equation 1$$

16. The screening method according to claim 1, wherein the cell is a prokaryotic microorganism.

17. A kit for carrying out the screening method according to claim 1, the kit comprising:
i. DNA encoding at least two membrane proteins and DNA encoding a sensor protein, or
ii. DNA encoding at least two sensor proteins and DNA encoding a membrane protein.

18. The screening method according to claim 2, wherein steps a) to f) are carried out for at least 20 membrane proteins with different amino acid sequences.

19. The screening method according to claim 2, wherein steps a) to f) are carried out for at least 50 membrane proteins with different amino acid sequences.

20. The screening method according to claim 2, wherein steps a) to f) are carried out for at least 10² to 10⁸ membrane proteins with different amino acid sequences.
